(19) United States
(12) Patent Application Publication
(10) Pub. No.: US 2023/0022259 A1
(43) Pub. Date: Jan. 26, 2023

(54) MESENCHYMAL STEM CELLS FOR USE IN THE TREATMENT OF OSTEOARTHRITIS IN ANIMALS

(71) Applicant: Boehringer Ingelheim Veterinary Medicine Belgium, Evergem (BE)

(72) Inventors: Jan SPAAS, Evergem (BE); Sarah BROECKX, Evergem (BE); Glenn PAUWELYN, Evergem (BE); Charlotte BEERTS, Evergem (BE)

(21) Appl. No.: 17/857,393

(22) Filed: Jul. 5, 2022

(30) Foreign Application Priority Data
Jul. 8, 2021 (EP) 21184474.1

(51) Int. Cl. A61K 35/28; A61P 19/02

(52) U.S. Cl. CPC A61K 35/28; A61P 19/02

(57) ABSTRACT

Mesenchymal stem cells (MSCs) or a pharmaceutical composition comprising a therapeutically effective amount of MSCs can be used in the treatment of osteoarthritis in canines and felines. For example, the MSCs or pharmaceutical composition can be used in the treatment of lameness and/or joint pain in canines and felines diagnosed with or suffering from osteoarthritis. A pharmaceutical composition comprising MSCs can be isolated from peripheral blood.

### CROSS-REFERENCE TO RELATED APPLICATIONS

This application claims priority to European Patent Application No. EP21184474.1 filed Jul. 8, 2021, the disclosure of which is hereby incorporated by reference.

### FIELD OF THE INVENTION

The present invention also relates to mesenchymal stem cells for use in the treatment of osteoarthritis in canines and felines.

### BACKGROUND

Osteoarthritis (OA) is one of the most frequently occurring joint disorders in veterinary practice as a consequence of metabolic disturbance and inflammatory responses in the joints. It is prevalent in approximately 20% of the dogs older than one year and characterized by a progressive degeneration and remodeling of the synovial joints, leading eventually to chronic pain, discomfort, swelling of the joint and lameness. Further, nearly 40% of all cats show clinical signs of OA, and 90% of cats over age 12 show radiographic evidence of OA. Weight management, tailored exercise and medical treatments for OA such as non-steroidal anti-inflammatory drugs (NSAIDs) and corticosteroids are mainly focused on pain relief and treatment of the inflammatory reaction, but are not able to slow down the disease progression or reverse the pathological condition. Persistent high doses of certain older generation NSAIDs are associated with gastro-intestinal, renal and hepatic abnormalities. Corticosteroids provide a rather short pain relief and could possibly induce further cartilage damage. Therefore, there is a very high demand for effective and long-term treatment options in canine and feline OA.

Mesenchymal stem cells (MSCs) have been proposed as a potential alternative because of their immunomodulatory properties, that could suppress the inflammation process of OA, slow down its progression on a very short term and even cause a reversion of the sustained damage. Several canine studies have investigated their safety and efficacy in the treatment of osteoarthritis and showed very interesting results.

The majority of these canine studies are using autologous MSCs derived from adipose tissue or bone marrow (BM) administered by an intra-articular injection in the affected joint. However, because osteoarthritis often affects multiple joints in a dog and cats, this kind of MSC therapy is very expensive and time consuming. An intra-articular injection is an invasive procedure, which requires sedation, experience and a targeted diagnosis.

In some cases, the use of allogeneic or xenogeneic MSCs is a more favorable option as they offer a stringent selection of healthy and high quality stem cell donors. They allow the production of a ready-to-use product, avoiding the invasive harvesting and time-consuming cultivation of MSCs from each individual patient. Because of the relative low culture capacity of canine and feline MSCs, xenogeneic (e.g. human or equine) MSCs may advantageously be used, especially for commercial applications, such as for use in the treatment of OA in canines or felines. In addition, xenogeneic MSCs are free of transmissible species-specific pathogens.

In some cases, the use of native MSCs is a favorable option as they allow the production of a ready-to-use product, with minimum manufacturing and handling, thereby lowering cost of production.

There remains a need in the art for an improved use of MSCs to slow down the disease progression and/or even reverse the pathological condition of OA in the families of cats and dogs. The present invention targets at solving at least one of the aforementioned disadvantages.

### SUMMARY OF THE INVENTION

The present invention and embodiments thereof serve to provide a solution to one or more of above-mentioned disadvantages. To this end, the present invention relates to mesenchymal stem cells (MSCs) or a pharmaceutical composition comprising a therapeutically effective amount of MSCs for use in the treatment of osteoarthritis in canines and felines. In embodiments, said MSCs are intravenously administered. In embodiments, said MSCs are native MSCs. In embodiments, said MSCs being are xenogeneic MSCs. Preferred embodiments of the MSCs for use of the invention are shown herein. In a particularly preferred embodiment, said treatment is the treatment of lameness and/or joint pain in canines and felines diagnosed with or suffering from osteoarthritis.

In a second aspect, the present invention relates to a pharmaceutical composition comprising native, peripheral blood-derived MSCs, said MSCs are animal-derived, and present in a sterile liquid.

Intravenously administered MSCs may reach multiple joints compared to local intra-articularly injected MSCS which only reach the local joint and must be applied to each of the affected joint(s) separately. Intravenous administration is a non-invasive procedure and does not require sedation. Such invasive procedures and/or sedation which may involve risks, especially for older patients which already are at higher risk in developing osteoarthritis. Therefore, the systemic administration of MSCs via an intravenous (IV) injection offers substantial benefits in therapy application.

### DESCRIPTION OF FIGURES

FIG. 1 shows an overview of the lameness assessment in dogs for three time points: Day 0—the day of the treatment, Follow-up 1—three weeks post treatment, and Follow-up 2—six weeks post treatment with native MSCs according to an embodiment of the invention.

FIG. 2 shows an overview of the range of motion (ROM) scores in dogs for three time points: Day 0—the day of the treatment, Follow-up 1—three weeks post treatment, and Follow-up 2—six weeks post treatment with native MSCs according to an embodiment of the invention.

FIG. 3 shows an overview of the joint effusion scores in dogs for three time points: Day 0—the day of the treatment, Follow-up 1—three weeks post treatment, and Follow-up 2—six weeks post treatment with native MSCs according to an embodiment of the invention.

FIGS. 4a-4g show the course of lameness—(a), articular pain—(b), joint effusion (c) score, range of motion (d), mean maximum force (e), symmetry index (f) and mean force (g) for different groups of dogs treated with native MSCs according to an embodiment of the invention or with a control product (mean±SD).

FIG. 5 shows PBMC proliferation before (day −7, left) and after (day 28, right) treatment of dogs with surgical induced osteoarthritis with 100.000 ePB-MSCs (low dose), 300.000 ePB-MSCs (target dose) or 1.500.000 ePB-MSCs (high dose) according to an embodiment of the invention, or saline (control), in a mixed lymphocyte reaction (MLR) assay with concanavalin A stimulated canine peripheral blood mononuclear cells (PBMCs), *p-value<0.05.

FIG. 6 shows PBMC proliferation before (day −7, left) and after (day 126, right) receiving different dosages of ePB-MSCs according to an embodiment of the invention or a placebo (i.e. T1: placebo (control group), T2: single injection with the recommended dose (=300.000 ePB-MSCs), T3: single injection with 3× the recommended dose, T4: single injection with 5× the recommended dose, T5: repeated injection (n=3) with the recommended dose and T6: repeated injection (n=3) with 5× the recommended dose), in a mixed lymphocyte reaction (MLR) assay with concanavalin A (ConA) stimulated canine peripheral blood mononuclear cells (PBMCs).

FIG. 7 shows the TGF-β1 concentration (mean±SD) in the supernatants of the negative control, positive control and immunomodulation samples of an MLR assay.

FIG. 8 shows the TNF-alfa concentration (mean±SD) in the supernatants of the negative control, positive control and immunomodulation samples of an MLR.

FIG. 9 shows the PGE2 concentration (mean±SD) in the supernatants of the negative control, positive control and immunomodulation samples of an MLR assay.

FIG. 10 shows a boxplot visualization of HA concentration normalized for Day 0 at Day −21, Day 14, Day 28 and Day 42 (*p-value<0.017).

FIG. 11 shows a boxplot visualization of HA concentration (ng/mL) based on post-hoc analysis (*p-value<0.05). Filled boxes: controls; dotted boxes: cases.

FIG. 12 shows a boxplot visualization of PGE2 concentration normalized for Day 0 at Day −21, Day 14, Day 28 and Day 42 (*p-value<0.017).

### DETAILED DESCRIPTION OF THE INVENTION

The present invention concerns native MSCs for use in the treatment of osteoarthritis (OA) in canines and felines, wherein said MSCs may be administered by intravenous injection. One intravenous injection is a systemic administration which reaches multiple joints compared to a local intra-articular injection which must be applied to each of the affected joint(s) separately. In addition, intravenous injection is a non-invasive procedure and does not require sedation. Such invasive procedures and/or sedation may involve risks, especially for older mammalian patients which already are at higher risk in developing osteoarthritis. Therefore, the systemic administration of MSCs via an intravenous (IV) injection offers substantial benefits in therapy application.

### Definitions

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6, or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

The terms “mesenchymal stem cells”, “MSCs” or “mesenchymal stromal cells” refer to multipotent, self-renewing cells that express a specific set of surface antigens and can differentiate into various cell types, including but not limited to adipocytes, chondrocytes, and osteocytes when cultured in vitro or when present in vivo.

The term “isolated”, refers to both the physical identification and isolation of a cells from a cell culture or a biological sample, like blood, that can be performed by applying appropriate cell biology technologies that are either based on the inspection of cell cultures and on the characterization (and physical separation when possible and desired) of cells corresponding to the criteria, or on the automated sorting of cells according to the presence/absence of antigens and/or cell size (such as by FACS). In some embodiments, the terms “isolating” or “isolation” may comprise a further step of physical separation and/or quantification of the cells, especially by carrying out flow cytometry.

The term “in vitro” as used herein denotes outside, or external to, a body. The term “in vitro” as used herein should be understood to include “ex vivo”. The term “ex vivo” typically refers to tissues or cells removed from a body and maintained or propagated outside the body, e.g., in a culture vessel or a bioreactor.

The term “passage” or “passaging” is common in the art and refers to detaching and dissociating the cultured (mesenchymal stem) cells from the culture substrate and from each other. For sake of simplicity, the passage performed after the first time of growing the cells under adherent culture conditions is generally referred to as “first passage” (or passage 1, P1). The cells may be passaged at least one time and preferably two or more times. Each passage subsequent to passage 1 is referred to with a number increasing by 1, e.g., passage 2, 3, 4, 5, or P1, P2, P3, P4, P5, etc.

The term “cell medium” or “cell culture medium” or “medium” refers to an aqueous liquid or gelatinous substance comprising nutrients which can be used for maintenance or growth of cells. Cell culture media can contain serum or be serum-free. The cell medium may comprise or be supplemented with growth factors.

The term “growth factor” as used herein refers to a biologically active substance which influences proliferation, growth, differentiation, survival and/or migration of various cell types, and may effect developmental, morphological and functional changes in an organism, either alone or when modulated by other substances. A growth factor may typically act by binding, as a ligand, to a receptor (e.g., surface or intracellular receptor) present in cells.

“Autologous” administration of MSCs in the present context refers to MSCs from a donor being administered to a recipient, wherein both recipient and donor are the same.

“Allogeneic” administration of MSCs in the present context refers to MSCs from a donor being administered to a recipient, wherein both recipient and donor are of the same species, but are not the same.

“Xenogeneic” administration of MSCs in the present context refers to MSCs from a donor being administered to a recipient, wherein the recipient and the donor are from different species.

“Native MSCs” in the context of the present invention refers to MSCs which have not been exposed to a stimuli environment, such as inflammatory mediators. As used herein, the “inflammatory environment” or “inflammatory condition” refers to a state or condition characterized by (i) an increase of at least one pro-inflammatory immune cell, pro-inflammatory cytokine, or pro-inflammatory chemokine; and (ii) a decrease of at least one anti-inflammatory immune cell, anti-inflammatory cytokine, or anti-inflammatory chemokine.

The term “anti-inflammatory”, “anti-inflammation”, “immunosuppressive”, and “immunosuppressant” refers to any state or condition characterized by a decrease of at least one indication of localized inflammation (such as, but not limited to, heat, pain, swelling, redness, and loss of function) and/or a change in systemic state characterized by (i) a decrease of at least one pro-inflammatory immune cell, pro-inflammatory cytokine, or pro-inflammatory chemokine; and (ii) an increase of at least one anti-inflammatory immune cell, anti-inflammatory cytokine, or anti-inflammatory chemokine.

The “population doubling time” or “PDT” of current invention is to be calculated by the formula: PDT=T/(In(N_{f}/Nᵢ)/In(2)), whereby T is the cell culture time (in days) to reach 80% confluency, N_{f} is the final number of cells after cell detachment and whereby Nᵢ is the initial number of cells at time point zero.

By the term “anti-coagulant”, it is meant a composition that can inhibit the coagulation of the blood. Examples of anticoagulants used in the present invention include EDTA or heparin.

The term “buffy coat” in this invention, is to be understood as the fraction of non-coagulated blood, preferably obtained by means of a density gradient centrifugation, whereby the fraction is enriched with white blood cells and platelets.

The term “blood-inter-phase” is to be understood as that fraction of the blood, preferably obtained by means of a density gradient, located between the bottom fraction, mainly consisting of erythrocytes and polymorphonuclear cells, and the upper fraction, mainly consisting of plasma. The blood-interphase is the source of blood mononuclear cells (BMCs) comprising monocytes, lymphocytes, and MSCs.

The term “suspension diameter” as used herein, is understood as the mean diameter of the cells, when being in suspension. Methods of measuring diameters are known in the art. Possible methods are flow cytometry, confocal microscopy, image cytometer, or other methods known in the art.

The term “therapeutically effective amount” is the minimum amount or concentration of a compound or composition that is effective to reduce the symptoms or to ameliorate the condition of a disease.

The term “treatment” refers to both therapeutic, prophylactic or preventive measures to reduce or prevent pathological conditions or disorders from developing or progressing.

“Osteoarthritis” (OA), also referred to as “Degenerative Joint Disease” (DJD), is a progressively worsening inflammation of the joint caused by the deterioration of cartilage. In a healthy joint, cartilage acts as a cushion to allow the joint to move smoothly through its full range of motion. In cases of osteoarthritis, this cartilage cushion begins to break down because of factors such as age, injury, repetitive stress, or disease. The loss of this protective cushion results in pain, inflammation, decreased range of motion, and the development of bone spurs. While any joint in the body can develop osteoarthritis, the condition most commonly affects the limbs and lower spine. For animals such as dogs and cats, typical visual signs of OA may include stiffness, lameness, or difficulty getting up; lethargy; reluctance to run, jump, or play; weight gain; irritability or changes in behavior; pain when petted or touched; difficulty posturing to urinate or defecate, or having accidents in the house; and/or loss of muscle mass over the limbs and spine.

The terms “patient”, “subject”, “animal”, or “mammal” are used interchangeably and refer to a mammalian subject to be treated. Preferably, the mammal is a canine or a feline, such as a dog or a cat.

“Feline” or “felines” in the present invention refers to cats of the Felidae family. A member of this family is also called a felid. The living Felidae are divided in two subfamilies: the Pantherinae and Felinae. Pantherinae includes five Panthera and two Neofelis species, while Felinae includes the other 34 species in ten genera, amongst which domestic cats, cheetahs, servals, lynx′ and cougars.

“Canine” or “canines” in the present invention refers to dog-like carnivorans of the Canidae family. A member of this family is called a canid. There are three subfamilies found within the canid family, which are the extinct Borophaginae and Hesperocyoninae, and the extant Caninae. The Caninae are known as canines, and include domestic dogs, wolves, foxes, coyotes, jackals and other extant and extinct species.

“Mixed Lymphocyte Reaction (MLR)” assays are traditionally used to investigate if an external agent stimulates or suppresses T-cell proliferation. By using a MLR assay, the immunomodulatory properties of the MSCs can be investigated. For this MLR assay the responder T-cells are marked with a fluorescent dye which lights up green when it is exposed to a specific light frequency. These responder T-cells are then stimulated with a plant mitogen Concanavalin A (ConA) to induce or stimulate proliferation. ConA is an antigen-independent mitogen and can be used as an alternative T cell stimulus. This lectin is frequently used as a surrogate for antigen-presenting cells in T cell stimulation experiments. Concanavalin A irreversibly binds to glycoproteins on the cell surface and commits T cells to proliferation. This is a quick way to stimulate transcription factors and cytokine production. When the T-cells start to divide the dye is distributed over their daughter cells, so the dye is serially diluting with every cell division. Therefore, the amount of proliferation of T-cells can be measured by looking at the decrease of color. Thus, to investigate the immunomodulatory properties of the MSCs, these MSCs are added to the stimulated responder T-cells and co-incubated for several days. Appropriate positive and negative controls are included to see if the test is performed successfully. At the end of the incubation period, the amount of T-cell proliferation is measured using flow cytometry, enabling to see whether or not the MSCs suppressed the T-cell proliferation.

### DESCRIPTION

In a first aspect, the present invention relates to mesenchymal stem cells (MSCs) or a pharmaceutical composition comprising a therapeutically effective amount of MSCs for use in the treatment of osteoarthritis (OA) in canines and felines, or as a method for treating OA in canines and felines or for use in the preparation of a medicament for the treatment of OA in canines and felines, wherein said MSCs are preferably native and preferably intravenously administered.

MSCs have been proposed for use in the treatment of inflammatory-related diseases because of their immunomodulatory properties. These immunomodulatory properties could suppress the exaggerated inflammatory process of, amongst others, osteoarthritis (OA), slow down its progression on a very short term and even cause a reversion of the sustained damage. Previous (canine) studies have investigated their safety and efficacy in the treatment of osteoarthritis and showed very interesting results. The majority of these canine studies are using autologous MSCs derived from adipose tissue or bone marrow (BM) administered by an intra-articular injection in the affected joint. However, because OA often affects multiple joints in a dog-like and cat-like mammals, this kind of MSC therapy is very expensive and time consuming. In addition, an intra-articular injection is an invasive procedure, which requires sedation, experience and a targeted diagnosis.

Therefore, systemic administration of MSCs may be advantageously achieved via an intravenous (IV) administration, e.g. through injection or infusion, offers substantial benefits in therapy application.

Said canine and feline may be any dog-like carnivoran of the Canidae family, preferably of the Caninae subfamily, more preferably a domestic dog (Canis familiaris); or any cat of the Felidae family, preferably of the Felinae subfamily, more preferably a domestic cat (Fells catus).

In an embodiment, said MSCs for use are native. Such native MSCs have not first in vitro been exposed to a stimulating agent, such as inflammatory mediators or an inflammatory environment. Such inflammatory environment refers to a state or condition characterized by (i) an increase of at least one pro-inflammatory immune cell, pro-inflammatory cytokine, or pro-inflammatory chemokine; and (ii) a decrease of at least one anti-inflammatory immune cell, anti-inflammatory cytokine, or anti-inflammatory chemokine. The use of native MSCs is sometimes a favorable option as they allow the production of a ready-to-use product, with minimum manufacturing and handling, thereby lowering cost of production.

By preference, the MSCs have a cell size between 10 μm to 100 μm, more preferably between 15 μm and 80 μm, more preferably between 20 μm and 75 μm, more preferably between 25 μm and 50 μm. In an embodiment, the MSCs for use according to the current invention are selected by size by means of a filter system, wherein the cells are run through a double filtration step using a 40 μm filter. Double or multiple filtration steps are preferred. The latter provides for a high population of single cells and avoids the presence of cell aggregates. Such cell aggregates may cause cell death during the preservation of the cells by freezing and may all have an impact on further downstream applications of the cells. For instance, cell aggregates may higher the risk of the occurrence of a capillary embolism when administered intravenously.

The MSCs for use according to the present invention may originate from various tissues or body fluids, in particular from blood, bone marrow, fat tissue or amniotic tissue. Bone marrow harvesting of MSCs has been reportedly associated with haemorrhage, chronic pain, neurovascular injury, and even death. Adipose tissue as a source for MSCs is regarded as a safer option. However, harvesting of MSCs from adipose tissue still requires an incision in the donor animal, hence this is still an invasive procedure. MSCs derived from blood show similar morphology as MSCs derived from bone marrow and adipose tissue. As a consequence, by preference, the MSCs originate from blood, including but not limited to umbilical cord blood and peripheral blood. More preferably, the MSCs originate from peripheral blood. Blood is not only a non-invasive and painless source, but also simple and safe to collect and, consequently, easily accessible. The MSCs or blood comprising MSCs may originate from all mammals, including, but not limited to, humans, domestic and farm animals, zoo animals, sport animals, pet animals, companion animals and experimental animals, such as, for example, mice, rats, rabbits, dogs, cats, cows, horses, pigs and primates, e.g., monkeys and apes; especially horse, human, cat, dogs, rodents, etc. In an embodiment, said origin of is equine. In particular MSCs may be derived from peripheral blood, preferably equine peripheral blood, which allows multiple MSC collections per year with minimal discomfort or morbidities for the donor animal.

In some cases, the use of allogeneic or xenogeneic MSCs is a more favorable option as they offer a stringent selection of healthy and high-quality stem cell donors. They allow the production of a ready-to-use product, avoiding the invasive harvesting and time-consuming cultivation of MSCs from each individual patient. Because of the relative low culture capacity of canine and feline MSCs compared to for example equine or human MSCs, the use of xenogeneic (e.g. human or equine) MSCs is preferred above allogeneic canine or feline MSCs, especially for commercial applications, such as for use in the treatment of OA in canines or felines.

Therefore, in a particular embodiment the MSCs of the current invention may be used for allogeneic or xenogeneic administration to a subject. As already indicated, allogeneic or xenogeneic use allows a better control of the quality of the MSCs, as different donors may be screened, and the optimal donors may be selected. The latter is indispensable in view of preparing functional MSCs. This is in contrast to autologous use of MSCs, as in this case, quality of the cells is more difficult to be ensured. Nonetheless, autologous use may have his benefits as well. In one case, blood MSCs are isolated, for which blood from a donor was used who was later also recipient of the isolated MSCs. In another case, blood is used from donors in which the donor is preferably of the same family, gender or race as the recipient of the MSCs isolated from the blood of donors. In particular, these donors will be tested on common current transmittable diseases or pathologies, in order to avoid the risk of horizontal transmission of these pathologies or diseases through the stem cells. Preferably, the donors/donor animals are kept in quarantine. When using donor horses they can be, for example tested for the following pathologies, viruses or parasites: equine infectious anemia (EIA), equine rhinopneumonitis (EHV-1, EHV-4), equine viral arteritis (EVA), West Nile virus (WNV), African horse Sickness (AHS), dourine (Trypanosoma), equine piroplasmosis, glanders (malleus, glanders), equine influenza, Lyme borreliosis (LB) (Borrelia burgdorferi, Lyme disease).

In an embodiment, the MSCs for use of the present invention may be characterized by the presence of/are measured positive for one or more of the following markers CD29, CD44, CD90, CD105, vimentin, fibronectin, Ki67, CK18 or any combination thereof. In a further embodiment, the MSCs for use of current invention may be characterized by the presence of mesenchymal markers CD29, CD44 and CD90. By means of the latter, the purity of the obtained MSCs can be analyzed, and the percentage of MSCs can be determined.

CD29 is a cell surface receptor encoded by the integrin beta 1 gene, wherein the receptor forms complexes with other proteins to regulating physiological activities upon binding of ligands. The CD44 antigen is a cell surface glycoprotein involved in cell-cell interactions, cell adhesion and migration. In addition, is CD44 a receptor for hyaluronic acid and can also interact with other ligands such as osteopontin, collagens and matrix metalloproteinases (MMPs). The CD90 antigen is a conserved cell surface protein considered as a marker for stem cells, like MSCs. The MSCs of current invention being triple positive for CD29/CD44/CD90 enables the person skilled in the art for a fast and unambiguous selection of the MSCs and provides the MSCs biological properties which are of interest for further downstream applications.

In an embodiment, the MSCs for use of the current invention are characterized by the absence of/measure negative for Major Histocompatibility Complex (MHC) class II molecules, preferably all currently known MHC Class II molecules, classifying the cell as a cell that can be used in cellular therapy for mammalians, such as feline or canine cellular therapy. Even when the MSCs are partly differentiated, the MSCs remain negative for MHC class II molecules. Detecting presence or absence, and quantifying the expression of MHC II molecules can be performed using flow cytometry.

In another and further embodiment the MSCs measure negative for CD45 antigen, a marker for hematopoietic cells.

In an embodiment, the MSCs measure negative for both MHC class II molecules and CD45.

In a particularly preferred embodiment, the MSCs for use of the current invention measure positive for mesenchymal markers CD29, CD44 and CD90 and measure negative for MHC class II molecules and CD45.

MSCs in general express MHC Class I antigen on their surface. In a particular embodiment the MSCs for use of current invention have a low or undetectable level of the MHC Class I marker. In a most preferred embodiment said MSCs measure negative for MHC Class II markers and have a low or undetectable level of MHC Class I marker, wherein said cell exhibits an extremely low immunogenic phenotype. For the sake of the current invention, said low level should be understood as less than 25%, more preferably less than 15% of the total cells expressing said MHC I or MHC II. Detecting presence or absence, and quantifying the expression of MHC I and MHC II molecules can be performed using flow cytometry.

These immunological properties of the MSCs limit the ability of the recipient immune system to recognize and reject cells, preferably allogeneic or xenogeneic cells, following cellular transplantation. The production of factors by MSCs, that modulate the immune response together with their ability to differentiate into appropriate cell types under local stimuli make them desirable stem cells for cellular therapy.

In an embodiment, the MSCs for use of the invention, secrete immunomodulatory prostaglandin E2 cytokine when present in an inflammatory environment or condition.

Inflammatory environments or conditions are characterized by the recruitment of immune cells of the blood. Inflammatory mediators include prostaglandins, inflammatory cytokines such as IL-1β, TNF-α, IL-6 and IL-15, chemokines such as IL-8 and other inflammatory proteins like TNF-α, IFN-γ. These mediators are primarily produced by monocytes, macrophages, T-cells, B-cells to recruit leukocytes at the site of inflammation and subsequently stimulate a complex network of stimulatory and inhibitory interactions to simultaneously destruct and heal the tissue from the inflammatory process.

Prostaglandin E2 (PgE2) is a subtype of the prostaglandin family. PgE2 is synthesized from arachidonic acid (AA) released from membrane phospholipids through sequential enzymatic reactions. Cyclooxygenase-2 (COX-2), known as prostaglandin-endoperoxidase synthase, converts AA to prostaglandin H2 (PgH2), and PgE2 synthase isomerizes PgH2 to PgE2. As a rate-limiting enzyme, COX-2 controls PgE2 synthesis in response to physiological conditions, including stimulation by growth factors, inflammatory cytokines and tumor promoters.

In a particular embodiment, said MSCs present in an inflammatory environment secrete the soluble immune factor prostaglandin E2 (PgE2) in a concentration ranging between 10³ to 10⁶ picogram per ml to induce or stimulate MSC-regulated immunosuppression.

The PgE2 secretion of the MSCs in those specific concentration ranges stimulates anti-inflammatory processes in vitro and together with their ability to differentiate into appropriate cell types makes them desirable for cellular transplantation.

In a preferred embodiment the MSCs for use of the current invention measures:
positive for mesenchymal markers CD29, CD44 and CD90;
positive for one or more markers comprised in the group consisting of vimentin, fibronectin, Ki67, or a combination thereof;
negative for MHC class II molecules;
negative for hematopoietic marker CD45, and
preferably have a low or undetectable level of MHC Class I molecules, wherein said low level should be understood as less than 25%, more preferably less than 15% of the total cells expressing MHC I.

In a most preferred embodiment, the MSCs for use of the current invention measures:
positive for mesenchymal markers CD29, CD44 and CD90;
positive for one or more markers comprised in the group consisting of vimentin, fibronectin, Ki67, or a combination thereof;
negative for MHC class II molecules;
negative for hematopoietic marker CD45; and
preferably have a low or undetectable level of MHC Class I molecules, wherein said low level should be understood as less than 25%, more preferably less than 15% of the total cells expressing MHC I,
   wherein said cell secretes immunomodulatory PgE2 cytokine in a concentration ranging between 10³ to 10⁶ picogram per ml when present in an inflammatory environment or condition.

In another or further embodiment, the MSCs for use according to the invention, have an increased secretion of at least one of the molecules chosen from IL-6, IL-10, TGF-beta, NO or a combination thereof, and a decreased secretion of IL-1 when present in an inflammatory environment or condition, and compared to an MSC having the same characteristics but not being subjected to said inflammatory environment or condition.

In a preferred embodiment, the MSCs have an increased secretion of at least one of the molecules chosen of IL-6, IL-10, TGF-β, NO, or a combination thereof, and a decreased secretion of IL-1 when present in an inflammatory environment or condition. Comparison can be made with a mesenchymal stem cell having the same characteristics as presented above, but which is not subjected to said inflammatory environment or condition.

Preferably the MSCs have an increased secretion of PgE2 in combination with two or more of the abovementioned factors.

PgE2, IL-6, IL-10, TGF-B and NO help suppressing the proliferation and function of major immune cell populations like T cells and B cells. In addition, the MSCs express low levels of MHC class I molecules and/or are negative for MHC class II molecules on their surface, escaping immunogenic reactions. In addition, the MSCs of current can suppress the proliferation of white blood cells by their increased secretion of abovementioned factors, once again helping to avoid immunogenic reactions of the host.

In another or further embodiment the MSCs stimulate the secretion of PgE2, IL-6, IL-10, NO, or a combination thereof and/or suppress the secretion of TNF-α, IFN-γ, IL-1, IL-13, or a combination thereof in the presence of peripheral blood mononuclear cells (PBMCs). In another or further embodiment, the MSCs suppress the secretion of TGF-β1 in the presence of PBMCs.

In the inflammatory environment the MSCs secrete multiple factors that modulate the immune response of the host. In addition, the MSCs have the stimulatory effect to induce or stimulate the secretion of one or more factors selected from the group consisting of PgE2, IL-6, IL-10, NO, or a combination thereof. Next to the stimulatory effect of the MSCs on the PBMCs in an inflammatory environment, the MSCs also have an suppressive effect on the secretion of the PBMCs, resulting in a decrease of one or more factors selected from the group consisting of TNF-α, IFN-γ, IL-1, TGF-β1, IL-13, or a combination thereof. The MSCs have a regulatory effect in the inflammatory environment, making them useful in the treatment of all sorts of diseases, particularly disorders of the immune system.

In general, any technology for identifying and characterizing cellular markers for a specific cell type (e.g. mesenchymal, hepatic, hematopoietic, epithelial, endothelial markers) or having a specific localization (e.g. intracellular, on cell surface, or secreted) that are published in the literature may be considered appropriate for characterizing MSCs. Such technologies may be grouped in two categories: those that allow maintaining cell integrity during the analysis, and those based on extracts (comprising proteins, nucleic acids, membranes, etc.) that are generated using such cells. Among the technologies for identifying such markers and measuring them as being positive or negative, immunocytochemistry or analysis of cell culture media are preferred since these allow marker detection even with the low amount of cells, without destroying them (as it would be in the case of Western Blot or Flow Cytometry).

Immunomodulatory properties of MSCs may be assayed using an MLR assay. For this MLR assay responder T-cells are marked with a fluorescent dye which lights up green when it is exposed to a specific light frequency. These responder T-cells are then stimulated with a plant mitogen (ConA) to induce or stimulate proliferation. When the T-cells start to divide the dye is distributed over their daughter cells, so the dye is serially diluting with every cell division. Therefore, the amount of proliferation of T-cells can be measured by looking at the decrease of color. Thus, to investigate the immunomodulatory properties of the MSCs, these MSCs are added to the stimulated responder T-cells and co-incubated for several days. Appropriate positive and negative controls are included to see if the test is performed successfully. At the end of the incubation period, the amount of T-cell proliferation is measured using flow cytometry, enabling us to see whether or not the MSCs suppressed the T-cell proliferation.

Relevant biological features of the MSCs can be identified by using technologies such as flow cytometry, immunocytochemistry, mass spectrometry, gel electrophoresis, an immunoassay (e.g. immunoblot, Western blot, immunoprecipitation, ELISA), nucleic acid amplification (e.g. real time RT-PCR), enzymatic activity, omics-technologies (proteomics, lipidomics, glycomics, translatomics, transcriptomics, metabolomics) and/or other biological activity.

The MSCs of current invention may be derived by any standard protocol known in the art. In an embodiment, said MSCs may be obtained via a method wherein the MSCs are isolated from blood or a blood phase and wherein said cells are cultured and expanded in a basal medium, preferably a low glucose medium.

Basal medium formulation as known in the art include, but are not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, Medium 199, Waymouth's 10 MB 752/1 or Williams Medium E, and modifications and/or combinations thereof. Compositions of the above basal media are generally known in the art and it is within the skill of one in the art to modify or modulate concentrations of media and/or media supplements as necessary for the cells cultured. A preferred basal medium formulation may be one of those available commercially such as DMEM, which are reported to sustain in vitro culture of MSCs, and including a mixture of growth factors for their appropriate growth, proliferation, maintenance of desired markers and/or biological activity, or long-term storage.

Such basal media formulations contain ingredients necessary for mammal cell development, which are known per se. By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g. glucose, pyruvate, e.g., sodium pyruvate, acetate, e.g., sodium acetate), etc. It will also be apparent that many media are available as low-glucose formulations with or without sodium pyruvate.

Method for isolating MSCs from blood or a blood phase and culturing and expanding said cells are known in the art and for instance described in WO2014053418 or WO2014053420.

In an embodiment, such method for isolating MSCs from blood or a blood phase and culturing and expanding said cells in a low glucose medium may comprise the following steps:
a) the collection of one or more blood samples from donors, in a sample vial, coated with an anti-coagulant;
b) centrifuging the blood samples to obtain a 3-phase distribution, consisting of a plasma-phase, buffy coat, and erythrocytes phase;
c) collecting the buffy coat and loading it on a density gradient;
d) collecting of the blood-inter-phase obtained from the density gradient of step c);
e) isolating of MSCs from the blood-inter-phase by centrifugation;
f) seeding between 2.5×10⁵/cm² and 5×10⁵/cm² MSCs in culture and keeping them in a low glucose growth medium supplemented with dexamethasone, antibiotics and serum.

In an embodiment, anticoagulants may be supplemented to the MSCs. Non-limiting examples are EDTA or heparin.

The number of seeding is crucial to ultimately obtain a pure and viable population MSCs at an acceptable concentration, as a too dense seeding will lead to massive cell death during expansion and a non-homogenous population of MSCs and a too dispersed seeding will result in little or no colony formation of MSCs, so that expansion is not or hardly possible, or it will take too much time. In both cases the viability of the cells will be negatively influenced.

In a preferred embodiment of current invention, the MSCs have a high cell viability, wherein at least 90%, more preferably at least 95%, most preferably 100% of said cells are viable.

The blood-interphase is the source of blood mononuclear cells (BMCs) comprising monocytes, lymphocytes, and MSCs. By preference, the lymphocytes are washed away at 37° C., while the monocytes die within 2 weeks in the absence of cytokines necessary to keep them alive. In this way, the MSCs are purified. The isolation of the MSCs from the blood-inter-phase is preferably done by means of centrifugation of the blood-inter-phase, after which the cell pellet is washed at least once with a suitable buffer, such as a phosphate buffer.

In a further embodiment the MSCs of current invention are negative for monocytes and macrophages, both within a range between 0% and 7.5%.

In particular, the mesenchymal cells are kept at least 2 weeks in growth medium. Preferably, growth medium with 1% dexamethasone is used, as the specific characteristics of the MSCs are kept in said medium.

Following a minimum period of 2 weeks (14 days), preferably 3 weeks (21 days) MSC colonies will become visible in the culture bottles. In a subsequent step g) at least 6×10³ stem cells/cm² are transferred to an expansion medium containing low glucose, serum and antibiotics for the purpose of expanding the MSCs. Preferably, the expansion of the MSCs will occur in minimal five cell passages. In this way sufficient cells can be obtained. Preferably, the cells are split at 70% to 80% confluency. The MSCs can be maintained up to 50 passages in culture. After this the risk of loss in vitality, senescence or mutation formation occurs.

In a further embodiment, the population doubling time (PDT) between each passage during expansion of the MSCs should be between 0.7 and 3 days after trypsinization. Said PDT between each passage during expansion of the MSCs is preferably between 0.7 and 2.5 days after trypsinization.

In a preferred embodiment, the MSCs for use according to the invention have a spindle-shaped morphology. The morphological characterization of the MSCs of current invention classifies the cell as an elongated, fibroblast-like, spindle-shaped cell. This type of cell is distinct form other populations of MSCs with small self-renewing cells which reveal mostly a triangular or star-like cell shape and populations of MSCs with a large, cuboidal or flattened pattern with a prominent nucleus. The selection of MSCs with this specific morphological characteristic along with the biological markers enables the person skilled in the art to isolate the MSCs of current invention. A morphological analysis of cells can easily be performed by a person skilled in the art using phasecontrast microscopy. Besides, the size and granularity of MSCs can be evaluated using forward and side scatter diagram in flow cytometry or other techniques known by a person skilled in the art.

In another or further preferred embodiment, the MSCs have a suspension diameter between 10 μm and 100 μm. The MSCs for use of current invention have been selected based on size/suspension diameter. By preference, the MSCs have a cell size between 10 to 100 μm, more preferably between 15 and 80 μm, more preferably 20 and 75 μm, more preferably between 25 and 50 μm. Preferably, the selection of cells based on cell size occurs by a filtration step. For instance, MSCs with a cell concentration ranging between 10³ to 10⁷ MSCs per ml, wherein said cells are preferably diluted in low glucose DMEM medium, are selected by size by means of a filter system, wherein the cells are run through a double filtration step using a 40 μm filter. Double or multiple filtration steps are preferred. The latter provides for a high population of single cells and avoids the presence of cell aggregates. Such cell aggregates may cause cell death during the preservation of the cells by freezing and may all have an impact on further downstream applications of the cells. For instance, cell aggregates may higher the risk of the occurrence of a capillary embolism when administered intravenously.

In an embodiment, said therapeutically effective amount of MSCs is between 10⁵-10⁷ MSCs in said composition.

In a preferred embodiment, the MSCs for use according to the present invention are formulated for administration in a subject by means of intravenous injection or infusion.

In an embodiment, a therapeutically effective amount of MSCs is administered to the canine or feline patient, preferably a dose of 10⁵-10⁷ MSCs per patient is administered. In an embodiment, a single dose is administered.

The minimum therapeutically effective dose that yields a therapeutic benefit to a subject is at least 10⁵ of the MSCs per administration. Preferably, each administration is by intravenous injection and comprises between 10⁵ to 5×10⁵ MSCs per administration, wherein said MSCs preferably are native and/or xenogeneic.

In an embodiment, said MSCs are administered at least twice, at least three times, at least four times, at least five times, preferably with intervals.

In another or further embodiment, the treatment further comprises: multiple administrations of the MSCs or the composition comprising MSCs, for example multiple intravenous administrations, doses of 10⁵-10⁷ MSCs per canine or feline patient, wherein said multiple doses are administered at various time points, including but not limited to one or more of the following time points 1 day apart, 2 days apart, 3 days apart, 4 days apart, 5 days apart, 6 days apart, 7 days (1 week) apart, 2 weeks apart, 3 weeks apart, 4 weeks apart, 5 weeks apart, 6 weeks apart, 7 weeks apart, 8 weeks apart, 3 months apart, 6 months, 9 months apart, and/or 1 year apart. Preferably each dose is administered at least 2 weeks apart, more preferably at least 3 weeks apart, even more preferably at least 4 weeks apart, and most preferably at least 6 weeks apart.

In an embodiment, said composition comprises said MSCs present in a sterile liquid. A non-limiting example of such sterile liquid is a minimal essential medium (MEM), such as Dulbecco's Modified Eagle Medium (DMEM). Said sterile liquid should be safe for intravenous administration, e.g. via injection or infusion, to a mammalian patient.

As non-limiting examples, said sterile liquid is a minimal essential medium, such as a basal medium. Basal medium formulation as known in the art include, but are not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, Medium 199, Waymouth's 10 MB 752/1 or Williams Medium E, and modifications and/or combinations thereof. Compositions of the above basal media are generally known in the art and it is within the skill of one in the art to modify or modulate concentrations of media and/or media supplements as necessary for the cells cultured. A preferred basal medium formulation may be one of those available commercially such as DMEM, which are reported to sustain in vitro culture of MSCs, and including a mixture of growth factors for their appropriate growth, proliferation, maintenance of desired markers and/or biological activity, or long-term storage.

Such basal media formulations contain ingredients necessary for mammal cell development, which are known per se. By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g. glucose, pyruvate, e.g., sodium pyruvate, acetate, e.g., sodium acetate), etc. It will also be apparent that many media are available as low-glucose formulations with or without sodium pyruvate.

By preference, said composition comprises at least 75%, more preferably at least 80%, even more preferably at least 85%, most preferably at least 90% of single cells and whereby said single cells have a suspension diameter of between 10 μm and 100 μm, more preferably between 15 μm and 80 μm, more preferably between 20 μm and 75 μm, more preferably between 25 μm and 50 μm. As previously mentioned, the diameter of the cells as well as their single-cell nature is crucial for any downstream application, e.g. intravenous administration, and for the vitality of the cells.

By preference, said composition comprises at least 90% MSCs, more preferably it will comprise at least 95% MSCs, more preferably at least 99%, most preferably 100% MSCs.

The volume and concentration of the composition in the form of a sterile liquid comprising the MSCs is preferably adapted for intravenous injection. In an embodiment, the pharmaceutical composition may be administered to the animal in the form of a sterile liquid comprising, after final adjustment, the MSCs at a concentration of 10⁵-10⁷ cells per mL.

In an embodiment, with each intravenous injection or infusion, a therapeutically effective amount of MSCs is administered, preferably each injection or infusion comprises a dose of 10⁵ to 10⁷ of said MSCs.

In an embodiment, the pharmaceutical composition comprises a therapeutically effective of amount of MSCs of between 10⁵-10⁷ MSCs per mL, preferably 10⁵ to 10⁶ MSCs per mL, more preferably 10⁵-5×10⁵ MSCs per mL of said composition.

In an embodiment, a therapeutically effective amount of MSCs is administered to the canine or feline patient, preferably a dose of 10⁵-10⁷ MSCs per patient is administered. In an embodiment, a single dose is administered.

In an embodiment, said MSCs are administered at least twice, at least three times, at least four times, at least five times.

In an embodiment, one dosage of said composition has a volume of about 0.5 to 5 ml, preferably of about 0.5 to 5 ml, preferably of about 0.5 to 3 ml, preferably of about 0.5 to 2 ml, more preferably of about 0.5 to 1.5 ml, most preferably of about 1 ml. In another or further embodiment, one dosage of said composition has a volume of maximally about 5 ml, preferably maximally about 4 ml, more preferably maximally about 3 ml, more preferably maximally about 2 ml, most preferably said volume is about 1 ml. This amount is suitable for intravenous administration.

Said dosage may be formulated in a vial or in a pre-filled syringe.

In an embodiment, the composition further comprises components selected from the group consisting of platelet-rich plasma (PRP), hyaluronic acid, compositions based on hyaluronic acid, glycosaminoglycans, or compositions based on glycosaminoglycans, or any combination thereof. These are known to have additional beneficial functions during downstream applications of the composition according to the current invention. Mixing of the MSCs with such carrier substances may in some cases be desirable to increase the effectiveness of the composition or create a synergistic effect. For instance, said carrier substances aid in the homing capacities and immunomodulating effects of the MSCs in the cell composition. PRP, for example, a substance rich in growth factors, stimulate the stem cells after implantation. Preferably, both the stem cells and PRP are harvested from the same donors for compatibility reasons. Carrier substances can also be used to counteract gravity: stem cells follow the law of gravity and therefore have difficulties reaching higher lesions without a carrier in which they can migrate. In addition, the carrier substances themselves also have beneficial effects on the pathological environment in which they contribute to the tissue repair itself and also provide a good stem cell niche to help differentiation of the cells in this area. Examples of hyaluronic acid, glycosaminoglycans or compositions on this basis include OSTENIL®, OSTENIL® +, Adant® and Adequan®.

In an embodiment, the volume of the composition which is administered per injection to a patient is adapted in accordance with the patient's body weight. In another embodiment, a fixed dose of 10⁵-10⁷ MSCs per patient, preferably 10⁵ to 10⁶ MSCs, more preferably 10⁵-5×10⁵ MSCs, most preferably 3×10⁵ MSCs is administered.

The inventors have further discovered that a particularly effective treatment is achieved by a dosing regimen comprising at least two dosages of the MSCs for use or the pharmaceutical composition for use as described above in any of the embodiments.

Therefore, a further embodiment relates to a pharmaceutical composition for use in the treatment of osteoarthritis in canines and felines, wherein:
the treatment comprises a step of administering, preferably intravenously, a first amount of said composition comprising a total dose of 10⁵-10⁷ MSCs per patient, and
the treatment further comprises a step of administering, preferably intravenously, a second amount of said composition, said second amount comprising a second total dose of 10⁵-10⁷ MSCs t, wherein said MSCs preferably are native and/or xenogeneic, and
   wherein said second dose is administered 1 day after the first amount, 2 days after the first amount, 3 days after the first amount, 4 days after the first amount, 5 days after the first amount, 6 days after the first amount, 7 days (1 week) after the first amount, 2 weeks after the first amount, 3 weeks after the first amount, 4 weeks after the first amount, 5 weeks after the first amount, 6 weeks after the first amount, 7 weeks after the first amount, 8 weeks after the first amount, 3 months after the first amount, 6 months, 9 months after the first amount, and/or 1 year after the first amount. Preferably each dose is administered at least 2 weeks after the first amount, more preferably at least 3 weeks after the first amount, even more preferably at least 4 weeks after the first amount, and most preferably at least 6 weeks after the first amount.

In an embodiment, said second dose is identical to the first dose. In another embodiment, said second dose is lower than the first dose. In yet another embodiment, said second dose is higher than the first dose.

In an embodiment, a third, fourth and/or even a fifth amount of said composition may be administered, preferably intravenously, to said patient, wherein said third, fourth and/or fifth amount comprises a third, fourth and/or fifth total dose of 10⁵-10⁷ MSCs, wherein said MSCs preferably are native and/or xenogeneic.

In an embodiment, a sixth or more amount of said composition may be administered, preferably intravenously, to said patient, wherein said sixth or more amount comprises a sixth or more total dose of 10⁵-10⁷ MSCs, wherein said MSCs preferably are native and/or xenogeneic.

Many canines and/or felines diagnosed with or suffering from osteoarthritis show (visual) signs of lameness and/or joint pain. Therefore, the invention also relates to the MSCs or the pharmaceutical composition comprising a therapeutically effective amount of MSCs as described in any of the previous embodiments, which are used in the in the treatment of lameness and/or joint pain in canines and felines diagnosed with or suffering from osteoarthritis, or as a method for treating lameness and/or joint pain in canines and felines diagnosed with or suffering from osteoarthritis, or for use in the preparation of a medicament for the treatment of lameness and/or joint pain in canines and felines diagnosed with or suffering from osteoarthritis. Said MSCs or composition are preferably intravenously administered, and said MSCs are preferably native and/or xenogeneic.

Treatment of lameness and/or joint pain comprises the prevention, the reduction, the mitigation, the amelioration and/or the reversion of said lameness and/or joint pain in the feline diagnosed with or suffering from osteoarthritis.

As indicated before, typical visual signs of osteoarthritis in canines and felines, such as domestic dogs and cats, may include stiffness, lameness, or difficulty getting up; lethargy; reluctance to run, jump, or play; weight gain; irritability or changes in behavior; pain when petted or touched; difficulty posturing to urinate or defecate, or having accidents in the house; and/or loss of muscle mass over the limbs and spine.

As osteoarthritis often affects multiple joints, treatment of lameness and joint pain by intravenously administering MSCs or a pharmaceutical composition comprising MSCs offers substantial benefits in therapy in multiple joins at once, compared to intra-articular administration of MSCs, which is an invasive procedure, and which requires sedation, experience and a targeted diagnosis.

In an embodiment, the MSCs or pharmaceutical composition comprising MSCs are as described in any of the embodiments above.

In a second aspect, the present invention relates to a specific pharmaceutical composition comprising peripheral blood-derived MSCs. Said composition comprises native peripheral blood-derived MSCs, said MSCs are animal-derived, preferably mammal-derived (e.g., equine-derived), and present in a sterile liquid at a concentration of between 10⁵-10⁷ MSCs per mL of said composition, wherein one dosage of said composition has a volume of about 0.5 to 5 ml, wherein said MSCs measure positive for mesenchymal markers CD29, CD44 and CD90 and measure negative for MHC class II molecules and CD45, and wherein said MSCs have a suspension diameter between 10 μm and 100 μm.

In an embodiment, said pharmaceutical composition is intravenously administered. In a preferred embodiment, said MSCs are equine derived.

In an embodiment, said one dosage of said composition has a volume of about 0.5 to 5 ml, preferably of about 0.5 to 5 ml, preferably of about 0.5 to 3 ml, preferably of about 0.5 to 2 ml, more preferably of about 0.5 to 1.5 ml, most preferably of about 1 ml. In another or further embodiment, one dosage of said composition has a volume of maximally about 5 ml, preferably maximally about 4 ml, more preferably maximally about 3 ml, more preferably maximally about 2 ml, most preferably said volume is about 1 ml. This amount is suitable for intravenous administration.

In another or further preferred embodiment, the MSCs have a suspension diameter between 15 and 80 μm, more preferably 20 and 75 μm, more preferably between 25 and 50 μm.

A person of ordinary skill will appreciate that elements of the aspects of the MSCs or the pharmaceutical composition for use in the treatment of osteoarthritis, or of the MSCs or pharmaceutical composition for use in the treatment of lameness and/or joint pain in canines and felines diagnosed with or suffering from osteoarthritis as described above return in the aspect of the pharmaceutical composition of the invention.

Consequently, all aspects of the present invention are related. All features and advantages as described in one of the aspects as described above, can relate to any of these aspects, even if they are described in conjunction with a specific aspect.

The MSCs or the pharmaceutical composition comprising MSCs for use according to the current invention, possibly together with further components as described above, will by preference be frozen in order to allow long-time storage of the MSCs or composition. Preferably the MSCs or composition will be frozen at low and constant temperature, such as a temperature below −20° C. These conditions allow a save storage of the MSCs or composition, and enable the MSCs to keep their biological and morphological characteristics, as well as their high cell viability during storage and once thawed.

In a more preferred embodiment the MSCs or the pharmaceutical composition comprising MSCs for use of the invention can be stored for at least 6 months at a maximum temperature of −80° C., optionally in liquid nitrogen. A crucial factor in the freezing of the MSCs is a cryogenic medium, in particular comprising DMSO. DMSO prevents ice crystal formation in the medium during the freezing process, but may be toxic to the cells in high concentrations. In a preferred embodiment, the concentration of DMSO comprises up to 20%, more preferably up to 15%, more preferably the concentration of DMSO in the cryogen comprises 10%. The cryogenic medium further comprises low-glucose medium such as low glucose DMEM (Dulbecco's Modified Eagle Medium).

Afterwards, the MSCs or the pharmaceutical composition for use of the invention are preferably thawed before administration at a temperature around room temperature, preferably at a temperature between 20° C. and 37° C., more preferably at a temperature between 25° C. and 37° C., and in a time span of maximal 20 minutes, preferably maximal 10 minutes, more preferably maximal 5 minutes.

Furthermore, the MSCs or composition is preferably administered within 2 minutes after thawing, in order to safeguard the vitality of the MSCs.

The invention is further described by the following non-limiting examples which further illustrate the invention, and are not intended to, nor should they be interpreted to, limit the scope of the invention.

### EXAMPLES AND/OR DESCRIPTION OF FIGURES

The present invention will now be further exemplified with reference to the following examples. The present invention is in no way limited to the given examples.

### EXAMPLE 1: ePB-MSCs for Use in the Treatment of Osteoarthritis in Dogs

The objective of this study is to evaluate the potential of a single intravenous injection of ePB-MSCs (equine peripheral-blood derived MSCs) as treatment for dogs suffering from naturally occurring articular pain, unresponsive to the current standard therapies. Hereby, the clinical effect and safety of the treatment are evaluated.

Materials and Methods

Dogs

A total of 50 canine patients are treated with the investigational product (IVP) in multiple veterinary practices (n=10). Patient inclusion is restricted by the following inclusion criteria: joint pain in one or multiple joints for several days/weeks, non-responsiveness to conservative therapies, confirmed lameness, confirmed pain by anamnesis, joint pain associated signs confirmed by radiography (RX) or other imaging modalities and completion of Canine Brief Pain Inventory (CBPI) questionnaires including pain severity score (PSS)≥3 and pain interference score (PIS)≥3. Patients with following conditions and treatments are excluded from the study: sprains, pregnancy, other diseases that could influence the clinical study, PSS<3 and PIS<3, changes in dog's regular medical treatment, corticosteroid administrations within the washout period, or an ongoing corticosteroid treatment. All dogs are observed for uncommon behavior, posture and the occurrence of potential adverse events, such as worsening of lameness, joint distention or skin allergy at the injection site, at three evaluation points during the study (Day 0, 3 weeks and 6 weeks post-treatment). Evaluations are performed by a veterinarian with at least 5 years of practical experience in the field of canine orthopedics. Owners, who are well informed, are tasked to report the occurrence of potential adverse events in between evaluation points. All regular medical treatments are continued during the study. This animal study is approved by the ethics committee of Global Stem cell Technology.

Isolation and Cultivation of ePB-MSCs (IVP)

According to previously described methods, the ePB-MSCs are isolated from venous blood collected from the vena jugularis of one donor horse. Prior to cultivation of the ePB-MSCs, serum is tested for the presence of multiple transmittable diseases as described by Broeckx et al. 2012. Subsequently the stem cells are cultivated in a Good manufacturing practice (GMP)-certified production site according to GMP-guidelines until passage (P) 5 and characterized on viability, morphology, presence of cell surface markers and population doubling time. Evaluation of the presence (Cluster of Differentiation CD29, CD44 and CD90) and absence (Major Histocompatibility Complex (MHC) II and CD45) of specific cell surface markers is accomplished by using flow cytometry as previously described (Spaas et al., 2013). However, the detailed expression and secretion pattern has been previously described in WO 2020/182935. The cell viability is assessed using trypan blue. Afterwards, the cells are further cultivated until P10, trypsinized and resuspended at a final concentration of 300.000 cells/mL in Dulbecco's Modified Eagle Medium (DMEM) low glucose with 10% dimethylsulfoxide (DMSO). The ePB-MSCs are stored at −80° C. in cryovials until further use. Sterility of the final product is tested by the absence of aerobic bacteria, anaerobic bacteria, fungi, endotoxins and mycoplasma. All patients are treated with the same batch of ePB-MSCs.

Study Design

All included canine patients are injected with one vial of the IVP containing 1 mL of ePB-MSC suspension. The vial is thawed in the palm of a hand and intravenously injected. Subsequently, the dogs are clinically evaluated by an experienced veterinarian at three evaluation points (Day 0: day of treatment administration, Follow-up 1: 3 weeks post-treatment and Follow-up 2: 6 weeks post-treatment) and observed thoroughly by a well-informed owner at all times. At the evaluation points, the effect of the treatment is investigated and scored by an orthopedic examination, lameness evaluation, range of motion (ROM) determination (subjective scoring+goniometry measurement) and an evaluation of the impact on the general clinical condition (Table 1). The goniometer, to determine ROM in degrees, is applied by placing the center over the axis of the limb and the transparent arms aligned with the anatomic landmarks on the limb. The measured values are compared to normal values according to Jaegger et al. (2002) (Table 2). Furthermore, the pain severity, pain interference and quality of life are scored by the owners using the CBPI at each of the follow-up points according to Brown (2017). The CBPI consists of eleven questions with a scoring system ranging from 0 to 10. Hereby the first four questions are used for scoring severity of the pain, the next six questions are used to determine pain interference and the last question gauges the overall quality of life of the dog (Table 3). Finally, at all three time points, the veterinarian evaluated and scored articular heat, articular pain and joint effusion by palpation of the affected joints (Table 4).

TABLE 1
An overview of the scoring system used during the
orthopedic and general clinical examination.

| Parameter | Score | Definition |
|---|---|---|
| Lameness | 1 | Stands, walks and trots normally |
| | 2 | Stands normally, slight painful gait when trotting |
| | 3 | Stands normally, slight painful gait when walking |
| | 4 | Stands normally, evident painful gait when walking |
| | 5 | Stands abnormally, evident painful gait when walking |
| Range | 0 | No limitation of movement or crepitus |
| of motion | 1 | 10 to 20 percent decrease in range of motion, no |
| | | crepitus |
| | 2 | 10 to 20 percent decrease in range of motion with |
| | | crepitus |
| | 3 | 20 to 50 percent decrease in range of motion |
| | 4 | More than 50 percent decrease in range of motion |
| Clinical | 0 | Not affected |
| condition | 1 | Mildly affected |
| | 2 | Moderately affected |
| | 3 | Severely affected |
| | 4 | Very severely affected |

TABLE 2
Goniometry measurements of normal canine range of motion.

Range of motion

| Joint | Flexion | Extension |
|---|---|---|
| shoulder | 57° | 165° |
| elbow | 36° | 166° |
| stifle | 41° | 162° |
| hip | 50° | 162° |

TABLE 3
The canine brief pain inventory

| # | Description | Question | Scoring |
|---|---|---|---|
| 1 | Pain | Score the pain at its worst | 0-10 |
| | severity | in the last 7 days | |
| 2 | score (PSS) | Score the pain at its least | 0-10 |
| | | in the last 7 days | |
| 3 | | Score the pain at its average | 0-10 |
| | | in the last 7 days | |
| 4 | | Score the pain as it is right | 0-10 |
| | | now | |
| 5 | Pain | Score pain interference with | 0-10 |
| | interference | general activity | |
| 6 | score (PIS) | Score pain interference with | 0-10 |
| | | enjoyment of life | |
| 7 | | Score pain interference with | 0-10 |
| | | ability to rise to standing | |
| | | from lying down | |
| 8 | | Score pain interference with | 0-10 |
| | | ability to walk | |
| 9 | | Score pain interference with | 0-10 |
| | | ability to run | |
| 10 | | Score pain interference with | 0-10 |
| | | ability to climb stairs, curbs, | |
| | | doorsteps, etc. | |
| 11 | Overall | Score the dog's overall | Poor - Fair - |
| | quality | quality of life over the last | Good - Very |
| | of life | 7 days | good - |
| | | | Excellent |

TABLE 4
An overview of the joint assessment scoring system

| Parameter | Score | Definition |
|---|---|---|
| Articular heat | 0 | No increased temperature sensation |
| sensation | 1 | Slightly increased temperature sensation |
| | 2 | Moderate increased temperature sensation |
| | 3 | Severe increased temperature sensation |
| Articular pain | 0 | None |
| | 1 | Mild signs (dog turns head in recognition) |
| | 2 | Moderate signs (dog pulls limb away) |
| | 3 | Severe signs (dog vocalizes or becomes |
| | | aggressive) |
| | 4 | Dog will not allow palpation |
| Joint effusion | 0 | None |
| | 1 | Mild signs (only at site of injection) |
| | 2 | Moderate signs (mild swelling entire joint) |
| | 3 | Severe sings (severe swelling entire joint) |
| | 4 | Extreme (peri-articular swelling) |

Statistical Analysis

A nonparametric longitudinal analysis is performed using the nparLD package available in R (Version 3.6.3). This method is the nonparametric counterpart of the mixed model. Time is included as categorical fixed effects factors and dog (or injury within dog) as stratification factor. The main time effect is tested through a robust rank based test at the global 5% significance level. Furthermore, post hoc pairwise comparisons between the three time points are done using the nonparametric Wilcoxon rank sum test and adjusted for multiple comparisons by Bonferroni's technique (comparison wise significance level=0.05/3=0.0133). The nonparametric 95% confidence interval is also derived for the difference between time points.

Results

Dogs

From 5 of the 50 treated canine patients, data cannot be obtained for the Follow-up 1 and/or Follow-up 2 time point(s), leading to missing data over the whole study period. Furthermore, 9 dogs do not comply with the preset in- and exclusion criteria. Finally, one dog is euthanized before the end of the study due to stomach rupture caused by gastric dilatation volvulus. The remaining 35 dogs which complied with the preset in- and exclusion criteria and have a complete follow-up data set are included in the data analysis of this study. These dogs suffer from pain in one or more of the following joints: elbow, stifle or hip. From these 35 included patients, 18 dogs have one affected joint (elbow: 12, stifle: 4 and hip: 2), 11 dogs have bilateral affected joints (elbow: 1, stifle: 3, hip: 6 and shoulder: 1) and 6 dogs have multiple bilateral affected joints (elbow+hip: 1, stifle+hip: 3, elbow+stifle: 1 and elbow+stifle+hip: 1) (Table 5).

TABLE 5
Overview of included patients.

| | | | | | duration of | | affected joint(s) | | | |
|---|---|---|---|---|---|---|---|---|---|---|
| dog | age | | gender | breed | complaints | | shoulder | elbow | stifle | hip |
| 1 | 11 | y | MC | Tibetan terrier | 2 | years | 0 | 2 | 0 | 2 |
| 2 | 9 | y | MC | CV | 2 to 3 months | | 0 | 2 | 2 | 0 |
| 3 | 16 | y | M | Labrador Retriever | several years | | 0 | 1 | 0 | 0 |
| 4 | 15 | y | MC | Bouvier de Flandres | 8 | years | 0 | 0 | 2 | 0 |
| 5 | 9 | y | MC | Boxer | several years | | 0 | 0 | 0 | 1 |
| 6 | 14 | y | F | Jack Russell terrier | 2 | years | 0 | 0 | 2 | 2 |
| 7 | 2 | y | F | Labrador Retriever | 4 | months | 0 | 0 | 2 | 0 |
| 8 | 13 | y | F | Czech shephard | 2 | years | 0 | 0 | 2 | 2 |
| 9 | 8 | y | F | German shorthaired | 3 | years | 0 | 0 | 1 | 0 |
| | | | | pointer | | | | | | |
| 10 | 13 | y | MC | Border collie | 2 | months | 0 | 1 | 0 | 0 |
| 11 | 5 | y | FC | Rhodesian ridgeback | 1.5 | years | 0 | 1 | 0 | 0 |
| 12 | 6 | y | F | CV | 2 | weeks | 0 | 1 | 0 | 0 |
| 13 | 11 | y | F | Danish dog | 2 | years | 0 | 0 | 0 | 1 |
| 14 | 2 y 1 m | | F | Bernese Sennen dog | 1 year 2 months | | 0 | 1 | 0 | 0 |
| 15 | 1 y 2 m | | M | Belgian shepherd | 9 | months | 0 | 0 | 1 | 0 |
| 16 | 2 y 8 m | | F | American Bulldog | 10 | months | 0 | 1 | 0 | 0 |
| 17 | 12 y 2 m | | M | Groenendael | 3 | years | 0 | 2 | 2 | 2 |
| 18 | 3 y 3 m | | F | Rottweiler | 2 | years | 0 | 0 | 0 | 2 |
| 19 | 5 y 1 m | | F | German shaphard | 9 | months | 0 | 0 | 0 | 2 |
| 20 | 8 | y | F | Cocker Spaniel | more than | | 0 | 0 | 2 | 2 |
| | | | | | 3 years | | | | | |
| 21 | m.d. | | m.d. | m.d. | m.d. | | 0 | 2 | 0 | 0 |
| 22 | m.d. | | m.d. | m.d. | m.d. | | 0 | 1 | 0 | 0 |
| 23 | m.d. | | m.d. | m.d. | m.d. | | 0 | 0 | 0 | 2 |
| 24 | m.d. | | m.d. | m.d. | m.d. | | 0 | 0 | 0 | 2 |
| 25 | 5 | y | FC | Sint bernard | 3 | years | 0 | 0 | 0 | 2 |
| 26 | 11 | y | M | Fox terrier | 2 | years | 0 | 0 | 1 | 0 |
| 27 | 12 | y | F | Golden Retriever | 1 | year | 0 | 0 | 0 | 2 |
| 28 | 3 y 2 m | | M | American Stafford | 1 year 10 months | | 0 | 0 | 1 | 0 |
| | | | | terrier | | | | | | |
| 29 | 7 | y | M | Border collie | several years | | 2 | 0 | 0 | 0 |
| 30 | 1 | y | M | CV | 2 | years | 0 | 1 | 0 | 0 |
| 31 | 9 | y | M | Cocker Spaniel | several years | | 0 | 1 | 0 | 0 |
| 32 | 10 | y | M | Dobberman | several years | | 0 | 0 | 2 | 0 |
| 33 | 11 | y | MC | Golden Retriever | several years | | 0 | 1 | 0 | 0 |
| 34 | 8 | y | FC | Labrador Retriever | 2 | months | 0 | 1 | 0 | 0 |
| 35 | 9 | y | M | Border collie | 1 | year | 0 | 1 | 0 | 0 |

y: year; m: month; m.d.: missing data; M: male; F: female; MC: male castrated; FC: female castrated; CV: Canis vulgaris; 0: no joint affected; 1: unilateral joint affected; 2: bilateral joints affected.

Orthopedic Examination

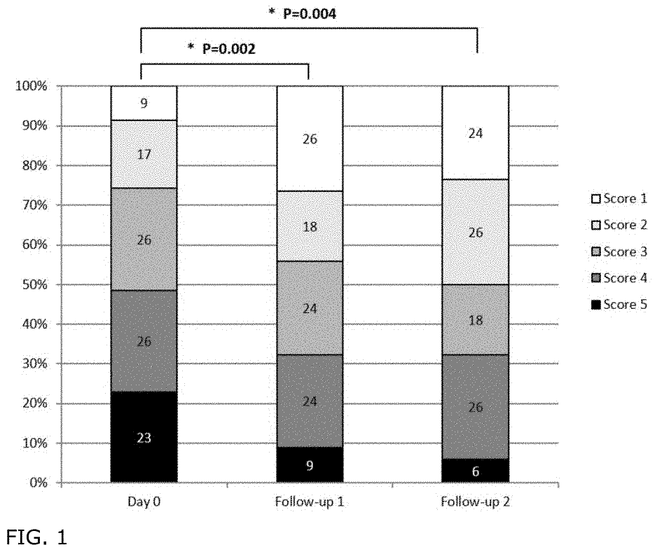

An overview of the lameness assessment for all three follow-up points is illustrated in FIG. 1. At the start of the study (Day 0), 23% of the dogs have a score of 5 on 5, 26% a score of 3 or 4 on 5, 17% of the dogs have a score of 2 on 5 and 9% of the dogs have a score of 1 on 5. Three weeks after treatment administration (Follow-up 1), lameness scores are significantly decreased with 44% of the dogs showing a lameness score of 1 or 2 on 5 (26% score 1 and 18% score 2) compared to 26% of the dogs with score 1 or 2 at baseline (Day 0) (P=0.002). Six weeks after treatment administration (Follow-up 2), 50% of the dogs have a lameness score of 1 or 2 on 5 (24% score 1 and 26% score 2), which is significantly better compared to baseline conditions (P=0.004). No significant differences in lameness score can be detected between Follow-up 1 and Follow-up 2 (P=0.484). Furthermore, assessment of lameness of individual patients show a decrease of lameness over a period of six weeks in 62% of the cases. The remaining 38% of the dogs show a stable (26%) or worsened (12%) degree of lameness six weeks post-treatment.

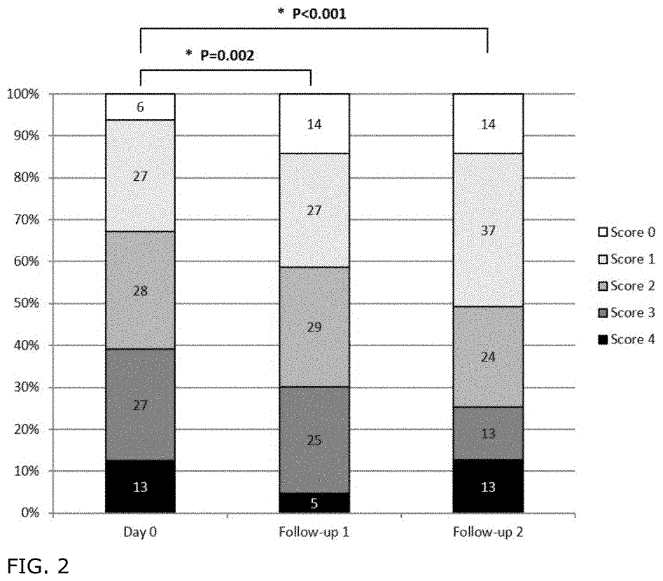

A second parameter tested during the orthopedic examination is the ROM of the affected joints. Results of the scoring are presented in FIG. 2. During the second follow-up, a significant decrease in impact on the range of motion is seen compared to baseline conditions (Day 0) (P<0.001). 50% of the dogs have a score of 0 or 1 compared to 33% on Day 0. A significant difference is also found between Day 0 and Follow-up 1 (P=0.002) but not between Follow-up 1 and Follow-up 2 (P=0.455). Additionally, an evaluation of ROM per joint six weeks post-treatment show an improvement of ROM in 34% of the joints. The ROM remains unchanged or worsened in respectively 61% and 5% of the joints.

The range of motion is also evaluated measuring the % change in degrees using a goniometer. Three weeks after treatment (Follow-up 1) the range of motion in degrees is significantly higher (P=0.001) with a median increase of 9.0 (95% CI: [3.5;16.0]) compared to baseline. After six weeks (Follow-up 2), the range of motion in degrees is significantly higher (P=0.004) with a median increase of 8.5 (95% CI: [2.5;13.5]) compared to baseline, but the two follow-up periods do not differ significantly (P=0.964).

Considering impact on clinical condition, no significant difference is found between all three time points.

Canine Brief Pain Inventory

During the three visits of the patients at the veterinary practice (Day 0, Follow-up 1 and Follow-up 2) the owners complete the CBPI, assessing the pain severity score, pain interference score and quality of life. This questionnaire reveals a significant decrease of PSS between Day 0 and Follow-up 1 (Median=−1; 95% CI: [−1.5,−0.5], P=0.005) but not between Day 0 and Follow-up 2 (Median=−1; 95% CI:[−1.0,0.0], P=0.073) and between the two follow-up periods. Concerning PIS, a significant decrease is found between Day 0 and Follow-up 1 (Median=−1.4; 95% CI: [−2.4,−0.9], P<0.001), between Day 0 and Follow-up 2 (Median=−2.15; 95% CI: [−3.1,−1.4], P<0.001) but not between Follow-up 1 and Follow-up 2 (Median=−0.5; 95% CI:[−1.0,0.1], P=0.016). Finally, an increase of quality of life is observed in 66% of the dogs over the six weeks study period after treatment administration. The quality of life remains the same or decrease during the six weeks study period in respectively 23% and 11% of the cases.

Joint Assessment

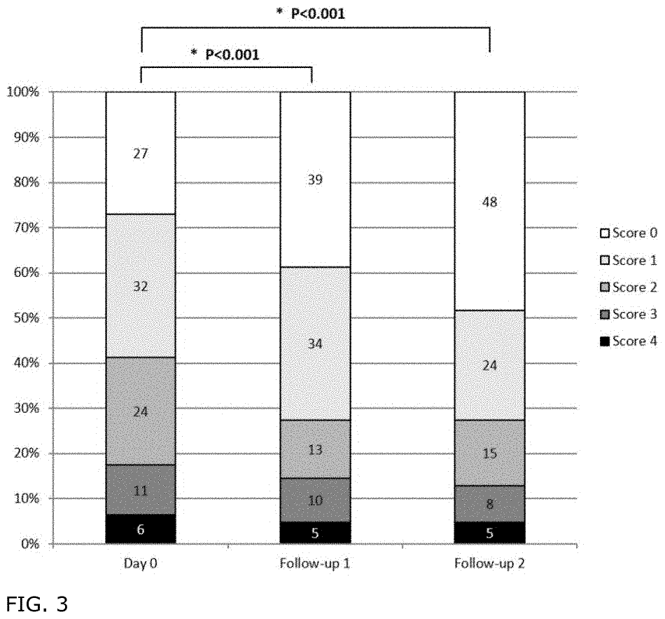

Finally, the joint assessment is performed after treatment administration. No significant differences can be found for heat sensation scores. Articular pain decrease significantly between Day 0 and Follow-up 1 (Median=−1.0; 95% CI:[−1.5,−0.5], P=0.005). No significant differences are found between Day 0 and Follow-up 2 (P=0.073) and between both follow-up periods (P=0.429). A decrease in joint effusion scores is found after treatment administration. The joint effusion score decrease significantly between Day 0 and Follow-up 1 (Median=−1.0; 95% CI: [−1.5;−1.0], P<0.001) and between Day 0 and Follow-up 2-(Median=−1.0; 95% CI: [−1.5;−1.0], P<0.001) but the two follow-up periods do not differ significantly (P=0.023) (FIG. 3). An evaluation of joint effusion per joint show a decrease amount of effusion in 38% of the joints six weeks after ePB-MSC administration. An equal or increased amount of joint fluid is detected in respectively 56% and 6% of the joints.

Discussion

To the best of our knowledge, this is the first study investigating the potential of intravenously injected xenogeneic equine peripheral blood-derived mesenchymal stem cells in dogs with articular pain. The stem cell therapy is well tolerated as there are no suspected adverse drug reactions or serious adverse events related to the treatment administration reported during this study. Furthermore, joint assessment shows no increase in articular heat sensation or articular pain. In contrast, a significant decrease of articular pain is detected three weeks post-treatment. Joint effusion decreases significantly three and six weeks after treatment administration.

Previous studies have investigated the safety and efficacy of MSCs in the treatment of OA when using autologous MSCs. However, this study is the first to describe the xenogeneic use of equine mesenchymal stem cells in dogs as treatment option for articular pain. Previously, the use of intra-articular administered equine chondrogenic induced MSCs in the treatment of OA was described, reducing lameness and pain in treated dogs according to the owner's evaluation. In the current study, native MSCs are intravenously injected instead of intra-articular which might have a substantial systemic anti-inflammatory effect, leading to reduced pain and inflammation in multiple affected joints. Furthermore, MSCs have shown to be able to migrate to inflammatory regions, contributing to an additional local anti-inflammatory effect. This type of administration also offers the advantage to be injected intravenously in a ready to treat formulation. This study demonstrates that the use of a single intravenous injection with ePB-MSCs is a promising and safe treatment of joint pain and lameness in dogs.

During the follow-up period, no deterioration and even a tendency to improvement of lameness and range of motion are detected on orthopedic examination. Lameness scores are significantly decreased at both follow-up time points compared to baseline conditions. A significantly decreased impact on motion range is found at Follow-up 1 and 2 compared to Day 0 measured with the scoring system as well as with the goniometer. No significant differences, however, can be found between both follow-up visits. The results of the canine brief pain inventory show an improvement of the pain experienced by the dogs. This is illustrated by a significant decrease in pain severity scores at the first follow-up visit and a significant decrease of interference scores at both follow-up visits compared to Day 0. No significant differences are found between Follow-up 1 and 2 although a further decrease can be observed. 66% of the dogs show an increase in quality of life.

Conclusion

In conclusion, the results of this study demonstrate that a single intravenous injection of ePB-MSCs is very well tolerated in canine patients suffering from joint pain and lameness. The joint pain and lameness do not aggravate after ePB-MSC injection and there is even a tendency to improvement.

### EXAMPLE 2: ePB-MSCs for Use in the Treatment of Osteoarthritis in Cats

A similar experiment is set up to evaluate the potential of a single intravenous injection of ePB-MSCs (equine peripheral-blood derived MSCs) as treatment for cats suffering from naturally occurring articular pain, unresponsive to the current therapies. Hereby, the clinical effect and safety of the treatment are evaluated.

Material and Methods

Cats

A group of feline patients is treated with the investigational product (IVP). Similar inclusion and exclusion criteria are applied. In particular, inclusion criteria are: joint pain in one or multiple joints for several days/weeks, non-responsiveness to conservative therapies, confirmed lameness, confirmed pain by anamnesis, joint pain associated signs confirmed by radiography (RX) or other imaging modalities. Exclusion criteria are: sprains, pregnancy, other diseases that can influence the clinical study, changes in cat's regular medical treatment, corticosteroid administrations within the washout period, or an ongoing corticosteroid treatment. All cats are observed for uncommon behavior, posture and the occurrence of potential adverse events, such as worsening of lameness, joint distention or skin allergy at the injection site, at three evaluation points during the study (Day 0, 3 weeks and 6 weeks post-treatment). Evaluations are performed by a veterinarian with at least 5 years of practical experience in the field of feline orthopedics. Owners, who are well informed, are tasked to report the occurrence of potential adverse events in between evaluation points. All regular medical treatments are continued during the study.

Isolation and Cultivation of ePB-MSCs (IVP), Study Design, and Statistical Analysis

ePB-MSCs are isolated and cultivated as in Example 1. The study design is very similar as for dogs in Example 1. All included feline patients are injected with one vial of the IVP containing 1 mL of ePB-MSC suspension. The vial is thawed in the palm of a hand and intravenously injected. Subsequently, the cats are clinically evaluated by an experienced veterinarian at three evaluation points (Day 0: day of treatment administration, Follow-up 1: 3 weeks post-treatment and Follow-up 2: 6 weeks post-treatment) and observed thoroughly by a well-informed owner at all times. At the evaluation points, the effect of the treatment is investigated and scored by an orthopedic examination, lameness evaluation, range of motion (ROM) determination (subjective scoring+goniometry measurement) and an evaluation of the impact on the general clinical condition. The goniometer, to determine ROM in degrees, is applied by placing the center over the axis of the limb and the transparent arms aligned with the anatomic landmarks on the limb. The measured values are compared to normal values. Furthermore, the pain severity, pain interference and quality of life are scored by the owners, in a similar manner as in Example 1. The statistical analysis method of Example 1 is applied.

Results

Six weeks after treatment, lameness scores are significantly better compared to baseline conditions. Over this period of six weeks, some individual patients show a decrease of lameness, while the degree of lameness is stable in others and only worsen in a minority of the cats. Secondly, the overall range of motion values are significantly improved three weeks after treatment. Six weeks post treatment, the joints of some of the individual patients show an improved ROM. Further, based on questionnaires complete by the cat's owners, a significant decrease in pain severity score is shown between baseline and three weeks post treatment. Also the pain interference score decreases between baseline and three weeks post treatment, and between baseline and six weeks post treatment. In addition, the quality of life of the majority of the cats improves over the six weeks of study, while this only d worsens for a small number of the cats. Finally, joint assessment after treatment shows that articular pain decreases after three weeks compared to baseline. In addition, also joint effusion scores decrease significantly after three and six weeks compared to baseline.

Discussion

To the best of our knowledge, this is the first study investigating the potential of intravenously injected xenogeneic equine peripheral blood-derived mesenchymal stem cells in cats with articular pain. The stem cell therapy proves to be well tolerated as there are no suspected adverse drug reactions or serious adverse events related to the treatment administration reported during this study. Furthermore, joint assessment shows no increase in articular heat sensation or articular pain. In contrast, a significant decrease of articular pain is detected three weeks post-treatment. Joint effusion decreases significantly three and six weeks after treatment administration.

Previous studies have investigated the safety of MSCs in felines. However, this study is the first to describe the xenogeneic use of equine mesenchymal stem cells in cats, intravenously injected, as treatment option for articular pain. In the current study, uninduced/native MSCs are intravenously injected instead of intra-articular which might have a substantial systemic anti-inflammatory effect, leading to reduced pain and inflammation in multiple affected joints. Furthermore, MSCs have shown to be able to migrate to inflammatory regions, contributing to an additional local anti-inflammatory effect. This type of administration also offers the advantage to be injected intravenously in a ready to treat formulation. This study demonstrates that the use of a single intravenous injection with ePB-MSCs is a promising and safe treatment of joint pain and lameness in cats.

The safety of the ePB-MSCs injection is currently evaluated based on clinical parameters. During the follow-up period, no deterioration and even a tendency to improvement of lameness and range of motion are detected on orthopedic examination. Lameness scores significantly decrease at both follow-up time points compared to baseline conditions. A significantly decreased impact on motion range is found at three and six weeks compared to Day 0. The results also show an improvement of the pain experienced by the cats. This is illustrated by a significant decrease in pain severity scores at the first follow-up visit and a significant decrease of interference scores at both follow-up visits compared to Day 0. The majority of cats also show an increase in quality of life.

Conclusion

In conclusion, the results of this study demonstrate that a single intravenous injection of ePB-MSCs is very well tolerated in feline patients suffering from joint pain and lameness. The joint pain and lameness does not aggravate after ePB-MSC injection and there is even a tendency to improvement.

### EXAMPLE 3: Dose Efficacy and Safety of IV Administered ePB-MSCs

In order to validate the optimal dosage for intravenous administration, a study is conducted for which 32 purpose bred dogs are divided into four treatment groups, receiving intravenously a dose of 0.2 times, 1 time or 5 times the recommended dose of 3×10⁵ ePB-MSCs, or a control injection without the ePB-MSCs of the present invention.

Method

Two weeks (day −14) before the treatment administration, osteoarthritis (OA) is surgically induced in the right stifle joint of each of 32 dogs by complete transection of the cranial cruciate ligament and creation of a bilateral cartilage defect on the weight bearing surfaces of the femoral condyles. On the day of treatment (day 0), the dogs are randomly divided into four treatment groups of eight dogs (T1, T2, T3 and T4). The dogs of treatment groups T1, T2 and T3 receive an IV (intravenous) injection with respectively 0.2 times (0.2 ml), 1 time (1 ml) and 5 times (5 ml) the recommended dose of 3×10⁵ ePB-MSCs. The dogs in treatment group T4, the control group, are intravenously injected with 5 ml 0.9% NaCl solution (Vetivex 9 mg/mL). All dogs undergo daily clinical observation to assess the occurrence of adverse events. Furthermore, orthopedic, clinical, hematological, pathological and histological parameters are evaluated during the study until 42 days post-treatment.

Results

General Clinical and Physical Assessment

During the entire study period, no adverse clinical events or alterations in general physical health are reported. Furthermore, no meaningful hematological and biochemical changes are detected according to regular blood analyses.

Orthopedic Assessment

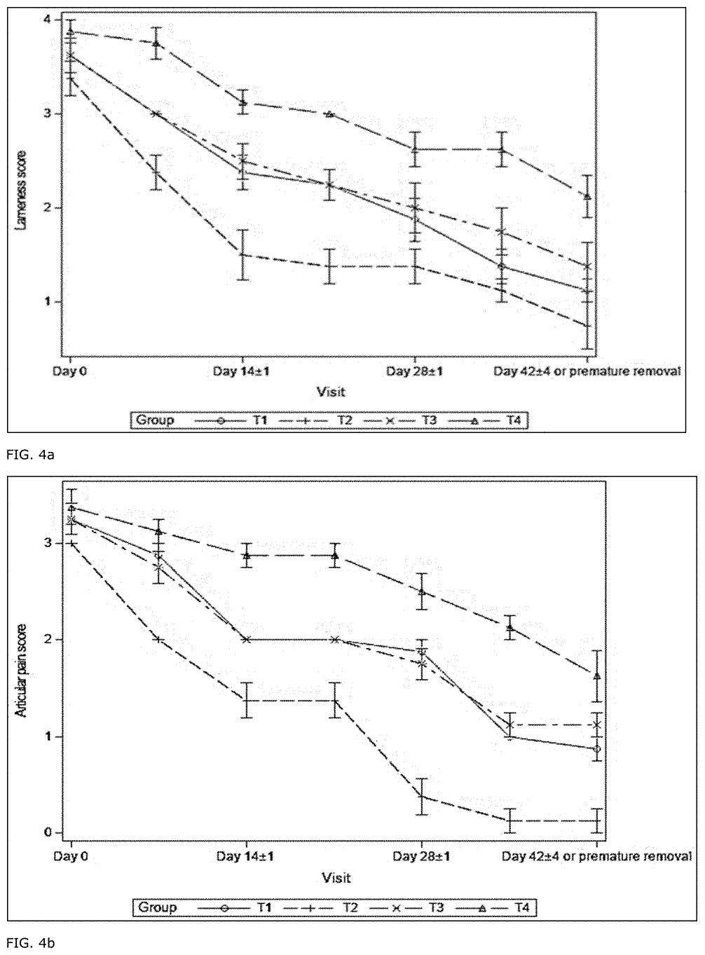

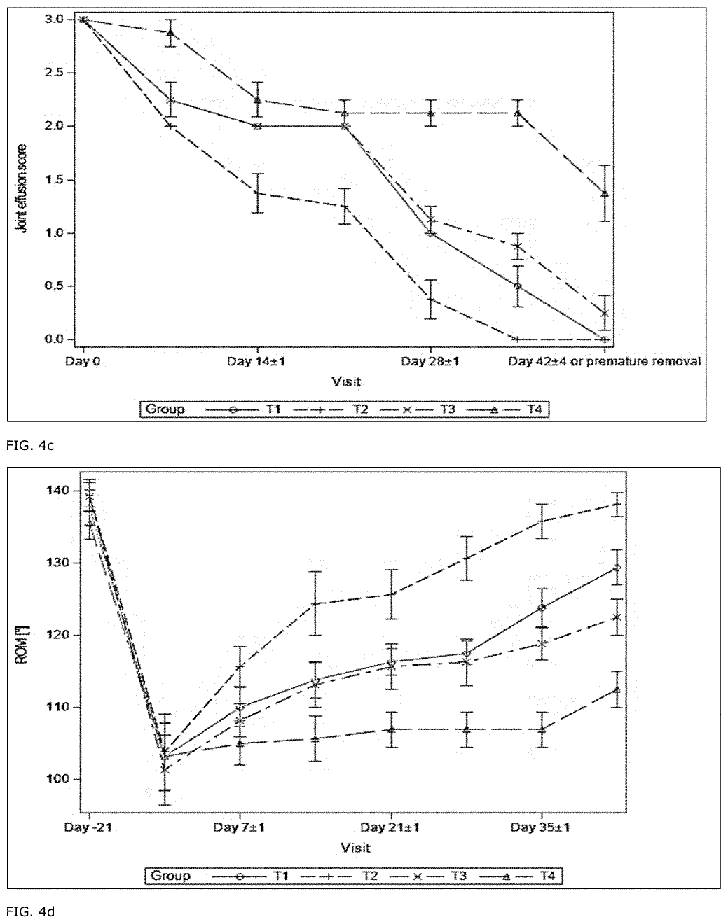

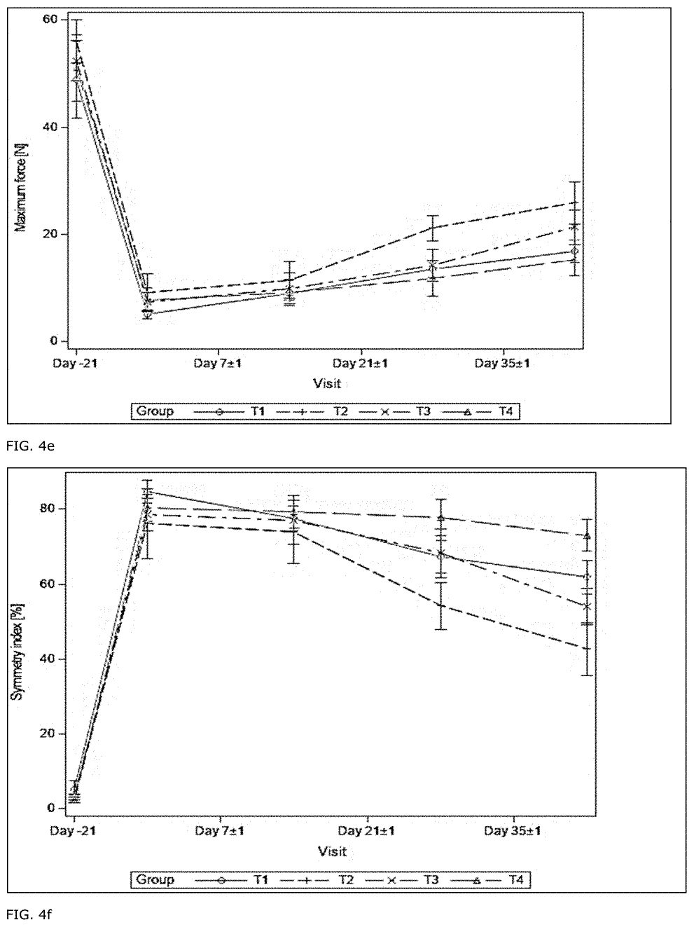

None of the animals show lameness, articular pain or joint effusion before OA introduction. At each of the post-baseline visits, fewer animals have no, mild or moderate articular lameness, articular pain and joint effusion in the control group (T4) compared to the three investigational veterinary product (IVP) groups (T1, T2, T3). In addition, a significant improvement from baseline at each of the post-baseline visits is found for all orthopedic scores in group T2. Furthermore, a significant reduction of the lameness score, articular pain score and joint effusion score is observed in group T2 on day 42±4 compared to T1 and T3 (FIGS. 4a, 4b and 4c). At day −21 and day 0, the mean range of motion (ROM) is similar in all groups. At each of the post-baseline visits from day 14±1 onwards, higher ROM values are found in the three IVP groups compared to group T4. Group T2 shows the highest increase of ROM at all post-baseline visits (FIG. 4d).

At day 0, the mean force (MF) and mean maximum force (MMF) was similar in all groups. However, at day 28±1, the MF and MMF was significantly different between groups (MF: P=0.031 and MMF: P=0.037) with higher means in the three IVP groups compared to group T4. The change of MF and MMF from day 0 to day 42±4 was significantly different between groups (MF: P=0.018 and MMF: P=0.045). Larger increases were found in each of the three IVP groups compared to group T4, with the highest increases in group T2 (FIGS. 4e and 4g). Similar results were found for the mean symmetry index (FIG. 4f).

CBPI Assessment

At inclusion, all animals have a pain severity score (PSS) and pain interference score (PIS) of zero. At day 0, the mean PSS and PIS scores are similar in all four groups. At each of the post-baseline visits, mean PSS and PIS scores are significantly lower in the three IVP groups compared to placebo (P≤0.001). Moreover, group T2 shows the highest score reduction for both PSS and PIS (PSS: −3.0±0.55; PIS: −3.1±0.72) compared to T1 (PSS: −1.9±0.35; PIS: −2.0±0.44), T3 (PSS: −1.6±0.38; PIS: −1.7±0.27) and T4 (PSS: −0.8±0.30; PIS: −0.28±0.33) on day 42±4 (data not shown). All animals have score 1 (=excellent) as quality of life score (QOL) at day −21 and score 4 (=fair) at day 0. At each of the post-baseline visits from day 7±1 onwards, more animals have a score 2 (=very good) or 3 (=good) in all three IVP groups compared to the group T4. Group T2 shows the highest percentage of animals with score 2 (75.0%) compared to T1 (12.5%) and T3 (0%) on day 42±4 and was significantly better than T4 (0%) (P≤0.001) (data not shown).

Pathology, Histology and Immunohistochemistry

At day 42±4, the highest incidences of score 1 for synovitis- and cartilages scores is observed in group T2 (87.5% and 87.5%) compared to group T1 (75.0% and 50.0%) and T3 (62.5% and 75.0%). The mean total score for each meniscus is lower in all three IVP groups compared to the group T4. This difference is significant for all group comparisons (P≤0.035), except for the medial meniscus in group T3 compared to group T4 (P=0.092). Group T2 shows the lowest mean score (0) for both menisci compared to group T1 (0.3±0.7) and T3 (1.0±1.2) (supplementary data). Histopathologic evaluations of the joint surface and the synovium show similar results for all groups. Furthermore, no significant differences can be found for the immunohistochemistry assessments of cartilage oligometric matrix protein (COMP), collagen type II, glycosaminoglycan and von Willebrand Factor (vWF) between the treatment groups. Finally, no ectopic tissue is found at the injection site, medial femoral condyle (MFC), the lateral femoral condyle (LFC), the medial tibial plateau and the lateral tibial plateau.

### EXAMPLE 4: Target Animal Safety

In order to assess safety of the single and repeated IV administration of ePB-MSCs to dogs, several clinical safety parameters are assessed in the present Example.

Methods

48 healthy purpose-bred beagles are randomly assigned to receive an intravenous injection with either the test item (n=40) or the reference item (n=8). Dogs treated with the test item, receive 3×10⁵ ePB-MSCs (1 mL) (n=8), 9×10⁵ ePB-MSCs (3 mL) (n=8) and 15×10⁵ ePB-MSCs (5 mL) (n=8) on day 0 (single injection treatment groups) or 3×10⁵ ePB-MSCs (1 mL) (n=8) and 15×10⁵ ePB-MSCs (5 mL) (n=8) on days 0, 42 and 84 (repeated injection treatment groups). Following injection, all dogs are evaluated for 252 days. All dogs undergo daily clinical observation to assess the occurrence of adverse events. Blood and urine samples are taken for haematological, coagulation and biochemical analysis. At the end of the study period, all animals are euthanized for a thorough necropsy and histology and to analyze the retention of ePB-MSCs.

Results

No overt differences in clinical safety parameters are observed between the control group and the treatment groups, with the exception of the temporary reticulocyte increase in several MSC treated groups. However, this is not associated with any adverse event and overall means are within reference range. None of the dogs show any clinical abnormalities during physical examinations, clinical and injection site observations. No significant decrease of body weight occurred. No adverse events related to the study medication are observed and none of the laboratory results indicates any overt abnormalities during the study. Based on literary research, abnormal findings (other than the reticulocytosis) throughout the study can be considered incidental and unrelated to the test item. No ectopic tissue is observed during necropsy and histopathologic evaluation. In addition, all (mild) abnormalities observed are unlikely to be related to the Test Item. PCR analysis found the eMSCs to be absent in the analyzed samples, indicating that the MSCs do not reside long term in the tissues or circulation.

Conclusion

The test item is shown to be safe for intravenous use/administration.

### EXAMPLE 5: Mixed Lymphocyte Reaction (MLR) in Dogs Before and After Treatment with ePB-MSCs

Set-Up:

To investigate the cellular immune response after inducing OA in dogs, before and after injecting the dogs with ePB-MSCs, a mixed lymphocyte reaction (MLR) assay was performed with peripheral blood mononuclear cells (PBMCs) isolated from the dogs. In order to confirm the immunomodulatory properties of the ePB-MSCs in dogs, ePB-MSCs are co-incubated with concanavalin A (conA) stimulated canine peripheral blood mononuclear cells (PBMCs) in a mixed lymphocyte reaction (MLR) assay. Consequently, PBMC proliferation (%) is evaluated using flow cytometry using Carboxyfluorescein succinimidyl ester 7-aminoactinomycin D (CFSE-7AAD) labeling. This assay is performed before and after treatment for all dogs included in two different clinical studies (proof of concept and safety study).

The 32 dogs in the proof of concept study are allocated to four treatment groups (i.e. low dose (T1) (100.000 ePB-MSCs), standard dose (T2) (300.000 ePB-MSCs) and high dose (T3) (1.500.000 ePB-MSCs), and a control group with saline (T4)) two weeks after induction of a surgical OA model. For this, the anterior cruciate ligament (ACL) cartilage defect model which leads to joint degeneration and cartilage defect, is used. The 48 dogs included in the safety study are divided into 6 equal groups of 8 dogs and received different dosages of the ePB-MSCs or a placebo (i.e. T1: placebo (control group), T2: single injection with the recommended dose (=300.000 ePB-MSCs), T3: single injection with 3× the recommended dose, T4: single injection with 5× the recommended dose, T5: repeated injection (n=3) with the recommended dose and T6: repeated injection (n=3) with 5× the recommended dose).

Results:

Proof of Concept Study

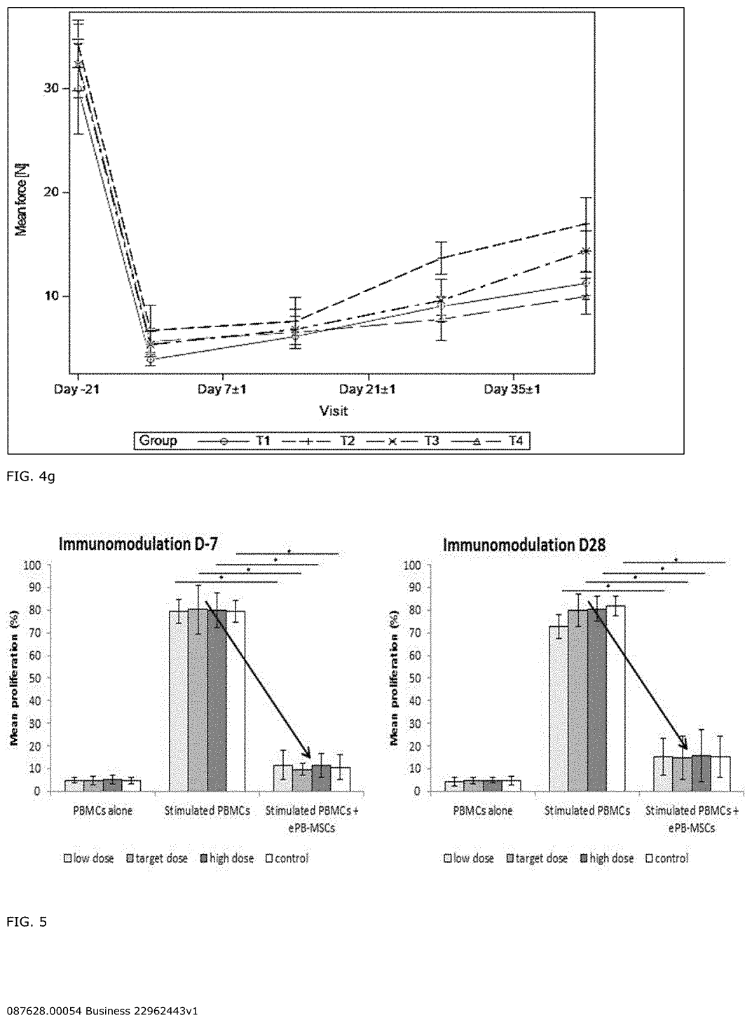

PBMC proliferation (%) of the co-incubation samples is significantly lower when compared to the positive control for all treatment groups (P<0.001), before (Day −7) and after treatment (Day 28 post-treatment). In addition, no significant difference is found between the PBMC proliferation (%) before treatment compared to after treatment (for each treatment group) (P≥0.061) (FIG. 5).

Safety Study

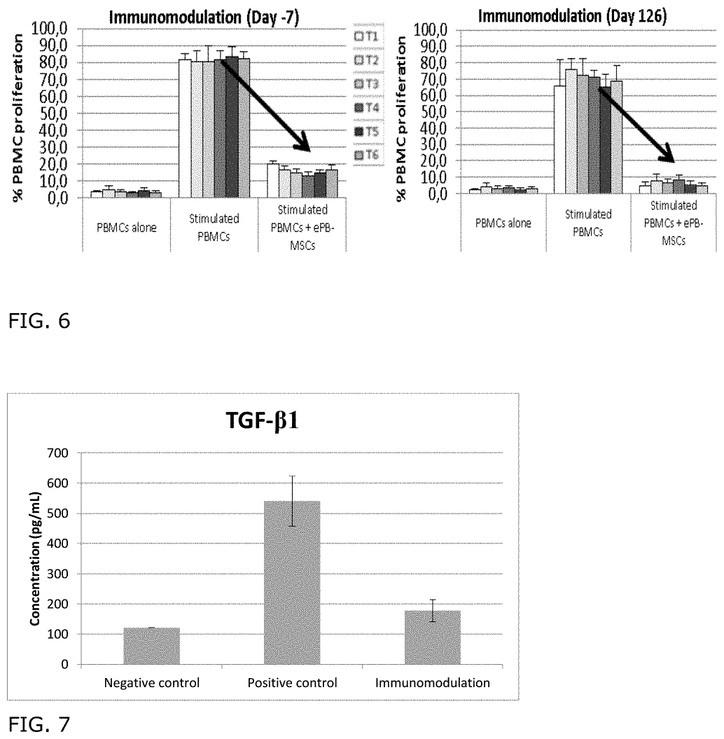

PBMC proliferation (%) of the co-incubation samples is significantly lower when compared to the positive control for all treatment groups (P≤0.002), before (Day −7) and after treatment (Day 126). In addition, the PBMC proliferation (%) for the co-incubation samples is decreased after treatment compared to before treatment. However, this is only significant for three out of six groups (FIG. 6).

Conclusion:

The PBMC proliferation for all treatment groups in both studies is significantly lower than the positive control before and after treatment, confirming the immunomodulatory capacities of ePB-MSCs to reduce the inflammatory response of stimulated canine PBMCs. In addition, strong immunomodulatory properties of the ePB-MSCs are found both before and after treatment. Furthermore, repeated injections do not reduce the immunomodulatory properties of the ePB-MSCs. On the contrary, these immunomodulatory properties are found to be significantly higher after intravenous injection in three out of six groups. The immunomodulatory properties confirm xenogeneic ePB-MSCs can be used in the treatment of osteoarthritis in dogs.

### EXAMPLE 6: Immunomodulatory and Pro-Inflammatory Markers in MLR Supernatants

Set-Up:

Immunomodulatory and pro-inflammatory markers are tested using commercial ELISA kits in the supernatants collected during the MLR assay co-incubating ePB-MSCs with conA stimulated canine PBMCs for 4 days. This in order to further investigate the immunomodulatory properties of equine peripheral blood-derived mesenchymal stem cells (ePB-MSCs) on canine PBMCs and to identify which paracrine factors secreted by the ePB-MSCs might be involved in this immunomodulation process.

Results:

Results show a large increase of TGF-β1 concentration in the positive control samples compared to the negative control sample. However, a large decrease is found in the immunomodulatory samples compared to the positive control, which is comparable to the negative control. The pro-inflammatory properties of TGF-β1 have previously been described and potentially induce or stimulate the differentiation of T-cells to the inflammatory T-helper 17 cell. These results indicate the production of TGF-β1 can be downregulated to baseline levels by the immunomodulatory properties of the ePB-MSCs (FIG. 7).

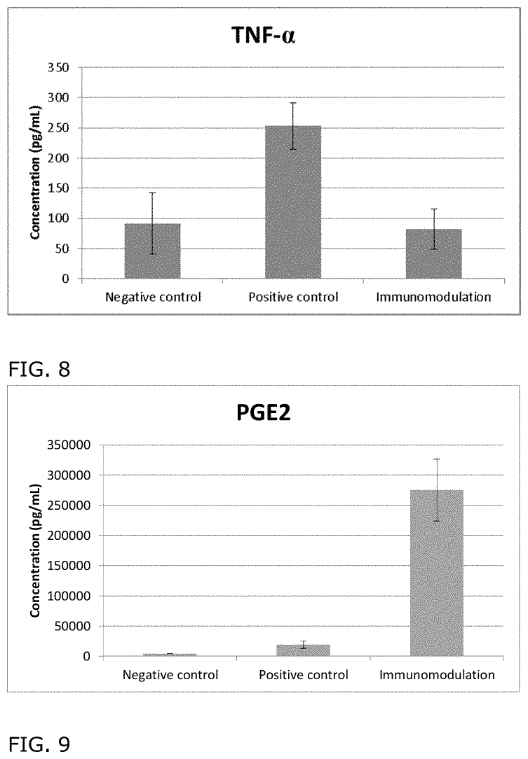

Furthermore, a significant increase of TNF-α concentration is found in the supernatants of the positive control samples compared to the negative control. This indicates, ConA stimulation of the canine PBMCs induces or stimulates the release of the pro-inflammatory cytokine TNF-α. ELISA results of the immunomodulation samples show the ePB-MSCs are able to lower the TNF-α concentration to the level of the negative control, indicating the ePB-MSCs are able to reverse this inflammatory environment by their immunomodulatory properties (FIG. 8).

Finally, PGE2 results indicate immunomodulation is correlated with a strong increase of PGE2 in the supernatants when comparing to the positive and negative control sample. These results indicate the importance of PGE2 in the immunomodulatory effect of ePB-MSCs (FIG. 9).

Conclusion:

ELISA on the immunomodulation supernatants samples shows that the ePB-MSCs are able to lower the TNF-α and TGF-β1 concentration to the level of the negative control, indicating the ePB-MSCs are able to reverse this inflammatory environment by their immunomodulatory properties. Furthermore, a strong increase of PGE2 is found in the immunomodulation supernatants samples compared to the positive and negative samples, which further strengthens the importance of PGE2 in the immunomodulatory effect of ePB-MSCs.

### EXAMPLE 7: Impact of IV ePB-MSCs Treatment on Inflammatory Markers and Cartilage Metabolites in Canine Blood

Set-Up:

In order to gain more insights in the in vivo immunomodulatory properties of the ePB-MSCs during the pathology of OA in dogs, different inflammatory markers and cartilage metabolites of interest are evaluated in the serum of 32 dogs before and after treatment at different time points (day −21, day 0, day 14, day 28 and day 48). The 32 dogs are allocated to four treatment groups (i.e. low dose (T1) (100.000 ePB-MSCs), standard dose (T2) (300.000 ePB-MSCs), high dose (T3) (1.500.000 ePB-MSCs) and control group (T4) with saline) two weeks after induction of a surgical OA model. For this, the anterior cruciate ligament (ACL) cartilage defect model which leads to joint degeneration and cartilage defect, is used. The inflammatory markers and cartilage metabolites investigated are transforming growth factor β1 (TGF-β1), prostaglandin E2 (PGE2), interferon-γ (IFN-γ), complement factor C3a, collagen type II cleavage (C2C) and hyaluronic acid (HA).

In a separate post-hoc analysis all dogs of this study re subdivided in controls and cases based on their synovitis and cartilage scores at the end of the study (Day 42) (controls: score≤2; case: score>2). Univariate statics were used to see if significant differences are found between these groups based on above mentioned markers.

Results:

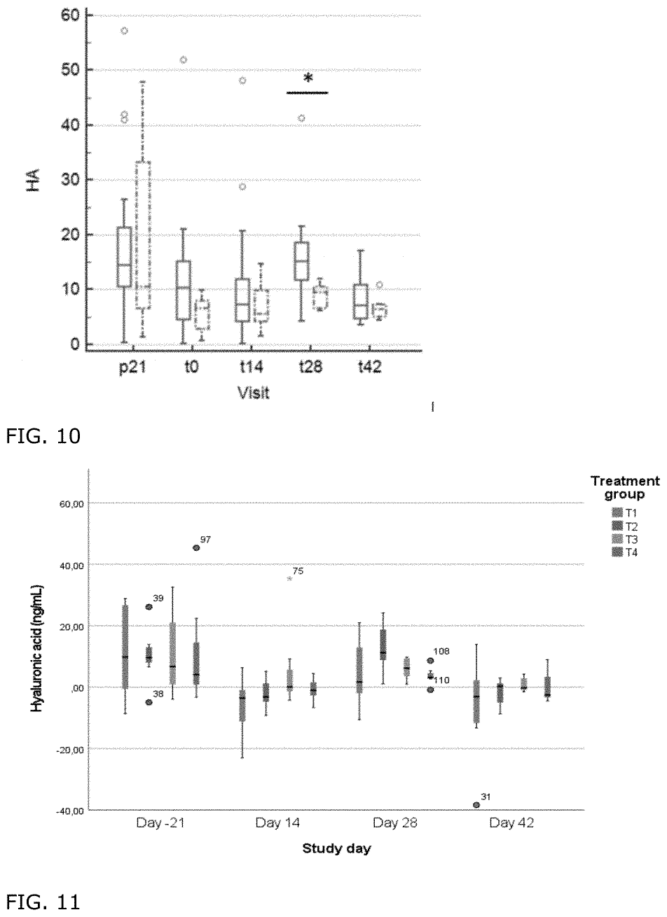

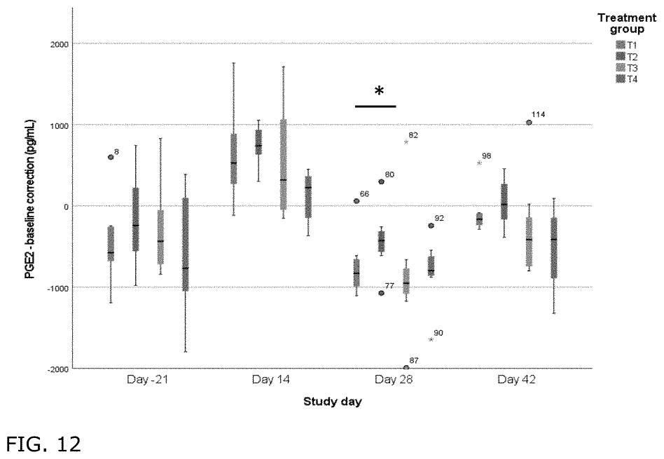

The results of the dogs treated with 300.000 ePB-MSCs (T2) show a significant increase of HA serum levels at Day 28 post-treatment compared to the control group (FIG. 10). Furthermore, the post-hoc analysis shows HA serum levels are also significant different between the case and control population (FIG. 11). In addition, higher serum levels of PGE2 are found in the dogs treated with 300.000 ePB-MSCs (T2) compared to the control group (T4) at all time points after treatment administration. However, this is only significantly different at Day 14 post-treatment (FIG. 12). No significant effects of ePB-MSCs administration are found on the other tested in vivo markers at the evaluated time points.

Conclusion:

The increased HA levels show ePB-MSCs are potentially causing a reduction in cartilage degradation and indicate a cartilage protective effect of the ePB-MSCs. This is strengthened by the post-hoc analysis showing a clear link between the cartilage and synovitis scoring and HA serum concentration. Furthermore, the increase in PGE2 levels confirms our in vitro findings that PGE2 is involved in the immunomodulatory mode of action of ePB-MSCs.

### EXAMPLE 8: Biodistribution of ePB-MSCs to Joints with OA

Set-Up:

A biodistribution study with ^{99m}Tc labelled ePB-MSCs in laboratory dogs using an OA model is set-up to investigate the homing behavior of the ePB-MSCs to the injury zone. Three purpose bred dogs undergo a surgery procedure to induce a canine OA model four days before start of biodistribution study. OA is induced in the right stifle joint of each dog by complete transection of the cranial cruciate ligament and creation of a bilateral cartilage defect on the weight bearing surfaces of the femoral condyles. Three days prior to surgery and four days after surgery, a power doppler examination is performed to evaluate the influence of the surgery on the vascularization in the joint. Next, the biodistribution of ePB-MSCs to the injured and healthy stifle joint 24h post-injection is examined using ^{99m}Tc labelled ePB-MSCs. The radioactivity in both stifle joints (control and lesion) is quantified using manually drawn regions of interest (ROI) on the lateral view of the whole-body scans. Relative uptake of ePB-MSCs in the lesion joint is expressed as fold increase in measured counts over the control lesion.

Main Results:

Investigating the biodistribution of ^{99m}Tc labelled ePB-MSCs in the canine OA model reveals a 13.0±3.9 fold higher uptake in the operated stifle joint compared to the control joint (n=3) 24 hours post-injection. Ultrasonic examination performed four days after the surgery indicates a mild increase in perfusion at the stifle joint of the operated limb while only a light increased perfusion is observed at the level of the healthy stifle joint (data not shown).

Conclusion:

These results demonstrate the natural homing behavior of ePB-MSCs to the injury zone after intravenous injection and provide more insights in their mode of action in canine OA treatment.

The present invention is in no way limited to the embodiments described in the examples and/or shown in the figures. On the contrary, methods according to the present invention may be realized in many different ways without departing from the scope of the invention.

1. Mesenchymal stem cells (MSCs) or a pharmaceutical composition comprising a therapeutically effective amount of MSCs for use in the treatment of osteoarthritis in canines and felines.

2. The MSCs or the pharmaceutical composition comprising the therapeutically effective amount of MSCs for use according to claim 1, wherein said MSCs are intravenously administered.

3. The MSCs or the pharmaceutical composition comprising the therapeutically effective amount of MSCs for use according to claim 1, wherein said MSCs are native.

4. The MSCs or the pharmaceutical composition comprising the therapeutically effective amount of MSCs for use according to claim 1, wherein said MSCs are derived from blood.

5. The MSCs or the pharmaceutical composition comprising the therapeutically effective amount of MSCs for use according to claim 1, wherein said MSCs are allogeneic or xenogeneic MSCs.

6. The MSCs or the pharmaceutical composition comprising the therapeutically effective amount of MSCs for use according to claim 1, wherein said MSCs are animal-derived.

7. The MSCs or a pharmaceutical composition comprising a therapeutically effective amount of MSCs for use according to claim 1, wherein a dose of 10⁵-10⁷ MSCs per canine or feline is administered.

8. The MSCs or the pharmaceutical composition comprising the therapeutically effective amount of MSCs for use according to claim 1, wherein a single dose is administered.

9. The MSCs or the pharmaceutical composition comprising the therapeutically effective amount of MSCs for use according to claim 1, wherein multiple doses are administered with each dose being administered at different time points.

10. The MSCs or the pharmaceutical composition comprising the therapeutically effective amount of MSCs for use according to claim 1, wherein one dosage of said composition has a volume of maximally about 5 ml.

11. The MSCs or the pharmaceutical composition comprising the therapeutically effective amount of MSCs for use according to claim 1, wherein said MSCs measure negative for MHC class II molecules and/or CD45.

12. The MSCs or the pharmaceutical composition comprising the therapeutically effective amount of MSCs for use according to claim 1, wherein said MSCs measure positive for mesenchymal markers CD29, CD44 and CD90 and measure negative for MHC class II molecules and CD45.

13. The MSCs or the pharmaceutical composition comprising the therapeutically effective amount of MSCs for use according to claim 1, wherein said MSCs secrete immunomodulatory prostaglandin E2 cytokine when present in an inflammatory environment or condition.

14. The MSCs or the pharmaceutical composition comprising the therapeutically effective amount of MSCs for use according to claim 1, wherein said MSCs have an increased secretion of at least one of the molecules chosen of IL-6, IL-10, TGF-β, NO, or a combination thereof; and/or a decreased secretion of IL-1 when present in an inflammatory environment or condition and compared to a cell having the same characteristics but not being subjected to said inflammatory environment or condition.

15. The MSCs or the pharmaceutical composition comprising the therapeutically effective amount of MSCs for use according to claim 1, wherein said MSCs stimulate the expression of PgE2, IL-6, IL-10, NO, or a combination thereof when in the presence of PBMCs and/or suppress the secretion of TNF-α, IFN-γ, IL-1, TGF-β, IL-13 or a combination thereof when in the presence of PBMCs.

16. The MSCs or the pharmaceutical composition comprising the therapeutically effective amount of MSCs for use according to claim 1, wherein the MSCs are present in a sterile liquid.

17. The MSCs or the pharmaceutical composition comprising the therapeutically effective amount of MSCs for use according to claim 1, wherein the MSCs or the composition further comprise components selected from the group consisting of platelet-rich plasma (PRP), hyaluronic acid, compositions based on hyaluronic acid, glycosaminoglycans, or compositions based on glycosaminoglycans or any combination thereof.

18. The MSCs or the pharmaceutical composition comprising the therapeutically effective amount of MSCs for use according to claim 1, wherein said treatment is the treatment of lameness and/or joint pain in canines and felines diagnosed with or suffering from osteoarthritis.

19. The MSCs or the pharmaceutical composition comprising the therapeutically effective amount of MSCs for use according to claim 18, wherein said MSCs or composition are intravenously administered.

20. A pharmaceutical composition comprising peripheral blood-derived MSCs, said MSCs are animal-derived and present in a sterile liquid at a concentration of between 10⁵-10⁷ MSCs per mL of said composition, wherein one dosage of said composition has a volume of about 0.5 to 5 ml, wherein said MSCs measure positive for mesenchymal markers CD29, CD44 and CD90 and measure negative for MHC class II molecules and CD45, and wherein said MSCs have a suspension diameter between 10 μm and 100 μm.
